(19) United States
(12) Patent Application Publication
(10) Pub. No.: US 2015/0125471 A1
(43) Pub. Date: May 7, 2015

(54) METHOD AND PHARMACEUTICAL COMPOSITION FOR USE IN THE TREATMENT AND DIAGNOTIC OF ANEMIA OF INFLAMMATION

(71) Applicants: INSERM (INSTITUT NATIONAL DE LA SANTE ET DE LA RECHERCHE MEDICALE), Paris (FR); UNIVERSITE PAUL SABATIER TOULOSE III, Toulouse (FR); CENTRE NATIONAL DE LA RECHERCHE SCIENTIFIQUE (CNRS), Paris (FR)

(72) Inventors: Marie-Paule Roth, Toulouse (FR); Hélène Coppin, Toulouse (FR)

(21) Appl. No.: 14/397,742

(22) PCT Filed: May 3, 2013
(86) PCT No.: PCT/EP2013/059235

§ 371 Date: Oct. 29, 2014

(30) Foreign Application Priority Data
May 3, 2012 (EP) 12305493.4

(51) Int. Cl. C07K 16/28; C12Q 1/68; G01N 33/74; C07K 16/26

(52) U.S. Cl. CPC C07K 16/2863; C07K 16/26; C12Q 1/6883; G01N 33/74; C07K 2317/76; C12Q 2600/158; G01N 2333/475; G01N 2800/7095; G01N 2800/22
USPC 424/158.1; 435/6.12; 436/501; 435/6.11; 506/9; 435/7.92

(57) ABSTRACT

The present invention relates to a compound which inhibits the binding of activin B to ALK3 receptor or a compound which is an inhibitor of activin B expression or ALK3 receptor expression for use in the treatment of anemia of inflammation. The invention also relates to a method for diagnosis anemia of inflammation in a patient comprising: determining the expression level of Activin B in a sample obtained from said patient; and comparing said expression level to a threshold value.

### FIELD OF THE INVENTION

The present invention relates to a compound which inhibits the binding of activin B to ALK3 receptor or a compound which is an inhibitor of activin B expression or ALK3 receptor expression for use in the treatment of anemia of inflammation. The invention also relates to a method for diagnosis anemia of inflammation in a patient comprising: determining the expression level of Activin B in a sample obtained from said patient; and comparing said expression level to a threshold value.

### BACKGROUND OF THE INVENTION

Anemia of inflammation occurs as a complication of an acute or chronic activation of the immune response. It is particularly common in hospitalized patients and in the elderly and has a negative impact on the recovery and survival of affected individuals (Weiss, G et al, 2005). Most chronic bacterial, fungal, viral or parasitic infections with systemic manifestations can cause anemia of inflammation, as well as rheumatologic disorders, systemic autoimmune disorders, inflammatory bowel disease, chronic kidney diseases, and some malignancies. The limitation of iron supply to erythropoiesis is a major factor in the development of this anemia. Attempts to treat anemia of inflammation with iron have generally been unsuccessful as iron is rapidly trapped into the macrophage compartment (Ganz, 2006; Ganz and Nemeth, 2009).

Anemia of inflammation appears to be caused, at least in part, by the induction of the iron-regulatory hormone, hepcidin (Nemeth et al., 2004a). Hepcidin acts by binding to the sole known iron export channel, ferroportin, found on the basolateral membrane of duodenal enterocytes, macrophages and hepatocytes, the cell types that export iron into plasma. Binding of hepcidin to ferroportin induces its internalization and degradation, which progressively inhibits iron efflux from these cells, leading to hypoferremia.

The fundamental mechanisms causing increased hepcidin production by the liver during inflammation are still imperfectly known, and the classical iron-induced BMP6/Smad signaling pathway appears dispensable. Iron overload induces expression of BMP6, a member of the transforming growth factor-β (TGF-β) superfamily. Binding of BMP6 to paired serine/threonine kinase type I and type II receptors results in phosphorylation of receptor-associated SMAD 1, 5 and 8 proteins which, after complexing with the common mediator protein SMAD4, translocate to the nucleus to bind specific regulatory elements in the promoter of the hepcidin gene, increasing hepcidin transcription. Hemojuvelin (HJV) acts as a BMP6 co-receptor and is as critical as BMP6 to hepcidin expression. Interestingly, although mice deficient for Bmp6 or for its coreceptor, hemojuvelin, have markedly reduced hepcidin synthesis, they are able to induce hepcidin production when challenged with lipopolysaccharide (LPS). Neither BMP6 nor HJV are therefore required for hepcidin upregulation in response to inflammation.

In contrast, STAT3 signaling is important for induction of hepcidin by inflammatory stimuli. The interaction of IL-6 or other mediators of the IL-6 family with its receptor results in phosphorylation of the intracellular signaling molecule STAT3. Phospho-STAT3 then dimerizes and is translocated to the nucleus where it interacts with a characterized response element in the hepcidin promoter. Interestingly however, transcriptional activation of hepcidin by IL-6 is abrogated in mice with liver-specific conditional knockout of Smad4 and a BMP-responsive element in the hepcidin promoter is required to control hepatic expression in response to IL-6. Furthermore, pharmacologic inhibition of BMP type I receptors by dorsomorphin or an optimized derivative, LDN-19318924, blocks induction of hepcidin expression by IL-6, turpentine, or group A streptococcal peptidoglycan-polysaccharide not only in cultured hepatoma-derived cells but also in zebrafish, mouse, or rat. Altogether, these observations provide evidence supporting a role not only for STAT3 but also for BMP/Smad signaling in the induction of hepcidin in response to inflammation. However, the molecule that activates BMP signaling in the inflammatory context is unknown.

### SUMMARY OF THE INVENTION

By using murine models, the inventors have identified the ligand of BMP I receptor that activates the BMP signalling. They show a dramatic induction of Inhbb mRNA, encoding activin βB-subunit, in the hepatocytes of mice challenged with lipopolysaccharide, slightly preceding the increase in Smad1/5/8 phosphorylation and Hamp mRNA. Activin B induces Smad1/5/8 phosphorylation in human hepatoma-derived cells and, synergistically with IL-6 and STAT3 signaling, markedly upregulates hepcidin expression.

Thus, the invention relates to a compound which inhibits the binding of activin B to ALK3 receptor or a compound which is an inhibitor of activin B expression or ALK3 receptor expression for use in the treatment of anemia of inflammation. The invention also relates to a method for diagnosis anemia of inflammation in a patient comprising: determining the expression level of Activin B in a sample obtained from said patient; and comparing said expression level to a threshold value.

### DETAILED DESCRIPTION OF THE INVENTION

A first object of the invention relates to a compound which inhibits the binding of activin B to ALK3 receptor or a compound which is an inhibitor of activin B expression or ALK3 receptor expression for use in the treatment of anemia of inflammation.

As used herein, the term “activin B” denotes a dimer composed of two identical or very similar beta subunits. Many functions have been found to be exerted by activin B, including roles in cell proliferation, differentiation, apoptosis, metabolism, homeostasis, immune response, wound repair, and endocrine function. An exemplary sequence for human activin B gene is deposited in the database under accession number NM_{—}002193. An exemplary sequence for human activin B protein is deposited in the UniProtKB/Swiss-Prot database under accession number NP_{—}002184.

As used herein, the term “ALK3 receptor” for “Activin receptor-Like Kinase 3 receptor” denotes a member of the bone morphogenetic protein (BMP) receptors family which is a transmembrane serine/threonine kinase. An exemplary sequence for human ALK3 receptor gene is deposited in the database under accession number NM_{—}004329. An exemplary sequence for human ALK3 receptor protein is deposited in the UniProtKB/Swiss-Prot database under accession number NP_{—}004320.

In one embodiment, the compound according to the invention may bind to activin B or ALK3 receptor and block the binding of activin B on ALK3 and block its effect on the BMP signalling pathway. To identify a compound able to block the interaction between activin B and ALK3 receptor, a test which demonstrates the effect of the compound on the induction of hepcidin gene as explained in the examples (FIG. 11) may be used.

Typically, the compound according to the invention includes but is not limited to a small organic molecule, an antibody, and a polypeptide.

In one embodiment, the compound according to the invention may be a low molecular weight compound, e. g. a small organic molecule (natural or not).

The term “small organic molecule” refers to a molecule (natural or not) of a size comparable to those organic molecules generally used in pharmaceuticals. The term excludes biological macromolecules (e. g., proteins, nucleic acids, etc.). Preferred small organic molecules range in size up to about 10000 Da, more preferably up to 5000 Da, more preferably up to 2000 Da and most preferably up to about 1000 Da.

In one embodiment, the compound according to the invention is an antibody. Antibodies directed against activin B or ALK3 receptor can be raised according to known methods by administering the appropriate antigen or epitope to a host animal selected, e.g., from pigs, cows, horses, rabbits, goats, sheep, and mice, among others. Various adjuvants known in the art can be used to enhance antibody production. Although antibodies useful in practicing the invention can be polyclonal, monoclonal antibodies are preferred. Monoclonal antibodies against activin B or ALK3 receptor can be prepared and isolated using any technique that provides for the production of antibody molecules by continuous cell lines in culture. Techniques for production and isolation include but are not limited to the hybridoma technique originally described by Kohler and Milstein (1975); the human B-cell hybridoma technique (Cote et al., 1983); and the EBV-hybridoma technique (Cole et al. 1985). Alternatively, techniques described for the production of single chain antibodies (see e.g., U.S. Pat. No. 4,946,778) can be adapted to produce anti-activin B or anti-ALK3 receptor single chain antibodies. Coumpounds useful in practicing the present invention also include anti-activin B or ALK3 receptor antibody fragments including but not limited to F(ab′)2 fragments, which can be generated by pepsin digestion of an intact antibody molecule, and Fab fragments, which can be generated by reducing the disulfide bridges of the F(ab′)2 fragments. Alternatively, Fab and/or scFv expression libraries can be constructed to allow rapid identification of fragments having the desired specificity to activin B.

Humanized anti-activin B or anti-ALK3 receptor antibodies and antibody fragments therefrom can also be prepared according to known techniques. “Humanized antibodies” are forms of non-human (e.g., rodent) chimeric antibodies that contain minimal sequence derived from non-human immunoglobulin. For the most part, humanized antibodies are human immunoglobulins (recipient antibody) in which residues from a hypervariable region (CDRs) of the recipient are replaced by residues from a hypervariable region of a non-human species (donor antibody) such as mouse, rat, rabbit or nonhuman primate having the desired specificity, affinity and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. Methods for making humanized antibodies are described, for example, by Winter (U.S. Pat. No. 5,225,539) and Boss (Celltech, U.S. Pat. No. 4,816,397).

Then, for this invention, neutralizing antibodies of activin B or ALK3 receptor are selected.

In one embodiment, the compound according to the invention is an anti-activin B antibody which neutralizes activin B or an anti-activin B fragment thereof which neutralizes activin B (see for example Ludlow H et al, 2008).

In a particular embodiment, the antibody according to the invention may be an antibody according to the patent application US2009317921.

In a particular embodiment, the antibody according to the invention may be an antibody according to the patent application US2009311252.

In a particular embodiment, the antibody according to the invention may be an antibody according to the patent application WO03006057.

In a particular embodiment, the antibody according to the invention may be the 46A/F compound (see for example Ludlow H et al, 2008).

In another embodiment, the compound according to the invention is an ALK3 receptor is an anti-ALK3 antibody which neutralizes ALK3 or an anti-ALK3 fragment thereof which neutralizes ALK3.

In a particular embodiment, the antibody according to the invention may be an antibody according to the patent application WO2010114860.

In one embodiment, the compound according to the invention is an aptamer. Aptamers are a class of molecule that represents an alternative to antibodies in term of molecular recognition. Aptamers are oligonucleotide or oligopeptide sequences with the capacity to recognize virtually any class of target molecules with high affinity and specificity. Such ligands may be isolated through Systematic Evolution of Ligands by EXponential enrichment (SELEX) of a random sequence library, as described in Tuerk C. and Gold L., 1990. The random sequence library is obtainable by combinatorial chemical synthesis of DNA. In this library, each member is a linear oligomer, eventually chemically modified, of a unique sequence. Possible modifications, uses and advantages of this class of molecules have been reviewed in Jayasena S. D., 1999. Peptide aptamers consists of a conformationally constrained antibody variable region displayed by a platform protein, such as E. coli Thioredoxin A that are selected from combinatorial libraries by two hybrid methods (Colas et al., 1996).

Then, for this invention, neutralizing aptamers of activin B or ALK3 receptor are selected.

In one embodiment, the compound according to the invention is a polypeptide.

In a particular embodiment the polypeptide is a functional equivalent of ALK3 receptor. As used herein, a “functional equivalent” of ALK3 receptor is a compound which is capable of binding to activin B, thereby preventing its interaction with ALK3 receptor. The term “functional equivalent” includes fragments, mutants, and muteins of ALK3 receptor. The term “functionally equivalent” thus includes any equivalent of ALK3 obtained by altering the amino acid sequence, for example by one or more amino acid deletions, substitutions or additions such that the protein analogue retains the ability to bind to activin B. Amino acid substitutions may be made, for example, by point mutation of the DNA encoding the amino acid sequence.

Functional equivalents include molecules that bind activin B and comprise all or a portion of the extracellular domains of ALK3 receptor. Typically, said functional equivalents may be the extracellular domains of ALK3 receptor expressed as Fc fusion protein as explained in the examples (FIG. 11).

In one embodiment, the polypeptide according to the invention is able to treat anemia of inflammation through its properties of decoy receptor.

By “decoy receptor”, is meant that the polypeptide according to the invention trap activin B and prevent its physiological effects on ALK3 receptor.

The functional equivalents include soluble forms of ALK3 receptor. A suitable soluble form of these proteins, or functional equivalents thereof, might comprise, for example, a truncated form of the protein from which the transmembrane domain has been removed by chemical, proteolytic or recombinant methods.

Preferably, the functional equivalent is at least 80% homologous to the corresponding protein. In a particular embodiment, the functional equivalent is at least 90% homologous as assessed by any conventional analysis algorithm such as for example, the Pileup sequence analysis software (Program Manual for the Wisconsin Package, 1996).

The term “a functionally equivalent fragment” as used herein also may mean any fragment or assembly of fragments of ALK3 receptor that binds to activin B. Accordingly the present invention provides a polypeptide capable of inhibiting binding of ALK3 receptor to activin B, which polypeptide comprises consecutive amino acids having a sequence which corresponds to the sequence of at least a portion of an extracellular domain of ALK3 receptor, which portion binds to activin B. In one embodiment, the polypeptide corresponds to an extracellular domain of ALK3 receptor. In another one embodiment, the polypeptide corresponds the extracellular domains of ALK3 receptor expressed as Fc fusion protein as explained in the examples (FIG. 11).

Functionally equivalent fragments may belong to the same protein family as the human ALK3 receptor identified herein. By “protein family” is meant a group of proteins that share a common function and exhibit common sequence homology. Homologous proteins may be derived from non-human species. Preferably, the homology between functionally equivalent protein sequences is at least 25% across the whole of amino acid sequence of the complete protein. More preferably, the homology is at least 50%, even more preferably 75% across the whole of amino acid sequence of the protein or protein fragment. More preferably, homology is greater than 80% across the whole of the sequence. More preferably, homology is greater than 90% across the whole of the sequence. More preferably, homology is greater than 95% across the whole of the sequence.

In one embodiment, the polypeptide according to the invention may be also a functional equivalent of activin B. As used herein, a “functional equivalent” of activin B is a compound which is capable of binding to ALK3 receptor, thereby preventing its interaction with the natural ligand activin B. The term “functional equivalent” includes fragments, mutants, and muteins of activin B. The term “functionally equivalent” thus includes any equivalent of activin B obtained by altering the amino acid sequence, for example by one or more amino acid deletions, substitutions or additions such that the protein analogue retains the ability to bind to ALK3 receptor. Amino acid substitutions may be made, for example, by point mutation of the DNA encoding the amino acid sequence.

The polypeptides of the invention may be produced by any suitable means, as will be apparent to those of skill in the art. In order to produce sufficient amounts of ALK3 receptor or functional equivalents thereof for use in accordance with the present invention, expression may conveniently be achieved by culturing under appropriate conditions recombinant host cells containing the polypeptide of the invention. Preferably, the polypeptide is produced by recombinant means, by expression from an encoding nucleic acid molecule. Systems for cloning and expression of a polypeptide in a variety of different host cells are well known.

When expressed in recombinant form, the polypeptide is preferably generated by expression from an encoding nucleic acid in a host cell. Any host cell may be used, depending upon the individual requirements of a particular system. Suitable host cells include bacteria mammalian cells, plant cells, yeast and baculovirus systems. Mammalian cell lines available in the art for expression of a heterologous polypeptide include Chinese hamster ovary cells. HeLa cells, baby hamster kidney cells and many others. Bacteria are also preferred hosts for the production of recombinant protein, due to the ease with which bacteria may be manipulated and grown. A common, preferred bacterial host is E coli.

In specific embodiments, it is contemplated that polypeptides used in the therapeutic methods of the present invention may be modified in order to improve their therapeutic efficacy. Such modification of therapeutic compounds may be used to decrease toxicity, increase circulatory time, or modify biodistribution. For example, the toxicity of potentially important therapeutic compounds can be decreased significantly by combination with a variety of drug carrier vehicles that modify biodistribution. In example adding dipeptides can improve the penetration of a circulating agent in the eye through the blood retinal barrier by using endogenous transporters.

A strategy for improving drug viability is the utilization of water-soluble polymers. Various water-soluble polymers have been shown to modify biodistribution, improve the mode of cellular uptake, change the permeability through physiological barriers; and modify the rate of clearance from the body. To achieve either a targeting or sustained-release effect, water-soluble polymers have been synthesized that contain drug moieties as terminal groups, as part of the backbone, or as pendent groups on the polymer chain.

Polyethylene glycol (PEG) has been widely used as a drug carrier, given its high degree of biocompatibility and ease of modification. Attachment to various drugs, proteins, and liposomes has been shown to improve residence time and decrease toxicity. PEG can be coupled to active agents through the hydroxyl groups at the ends of the chain and via other chemical methods; however, PEG itself is limited to at most two active agents per molecule. In a different approach, copolymers of PEG and amino acids were explored as novel biomaterials which would retain the biocompatibility properties of PEG, but which would have the added advantage of numerous attachment points per molecule (providing greater drug loading), and which could be synthetically designed to suit a variety of applications.

Those of skill in the art are aware of PEGylation techniques for the effective modification of drugs. For example, drug delivery polymers that consist of alternating polymers of PEG and tri-functional monomers such as lysine have been used by VectraMed (Plainsboro, N.J.). The PEG chains (typically 2000 daltons or less) are linked to the a- and e-amino groups of lysine through stable urethane linkages. Such copolymers retain the desirable properties of PEG, while providing reactive pendent groups (the carboxylic acid groups of lysine) at strictly controlled and predetermined intervals along the polymer chain. The reactive pendent groups can be used for derivatization, cross-linking, or conjugation with other molecules. These polymers are useful in producing stable, long-circulating pro-drugs by varying the molecular weight of the polymer, the molecular weight of the PEG segments, and the cleavable linkage between the drug and the polymer. The molecular weight of the PEG segments affects the spacing of the drug/linking group complex and the amount of drug per molecular weight of conjugate (smaller PEG segments provides greater drug loading). In general, increasing the overall molecular weight of the block co-polymer conjugate will increase the circulatory half-life of the conjugate. Nevertheless, the conjugate must either be readily degradable or have a molecular weight below the threshold-limiting glomular filtration (e.g., less than 60 kDa).

In addition, to the polymer backbone being important in maintaining circulatory half-life, and biodistribution, linkers may be used to maintain the therapeutic agent in a pro-drug form until released from the backbone polymer by a specific trigger, typically enzyme activity in the targeted tissue. For example, this type of tissue activated drug delivery is particularly useful where delivery to a specific site of biodistribution is required and the therapeutic agent is released at or near the site of pathology. Linking group libraries for use in activated drug delivery are known to those of skill in the art and may be based on enzyme kinetics, prevalence of active enzyme, and cleavage specificity of the selected disease-specific enzymes. Such linkers may be used in modifying the protein or fragment of the protein described herein for therapeutic delivery.

In another embodiment, the compound according to the invention is an inhibitor of activin B expression or ALK3 receptor expression.

Small inhibitory RNAs (siRNAs) can also function as inhibitors of activin B or ALK3 receptor gene expression for use in the present invention. Activin B or ALK3 receptor gene expression can be reduced by contacting a subject or cell with a small double stranded RNA (dsRNA), or a vector or construct causing the production of a small double stranded RNA, such that activin B or ALK3 receptor gene expression is specifically inhibited (i.e. RNA interference or RNAi). Methods for selecting an appropriate dsRNA or dsRNA-encoding vector are well known in the art for genes whose sequence is known (e.g. see for example Tuschl, T. et al. (1999); Elbashir, S. M. et al. (2001); Hannon, G J. (2002); McManus, M T. et 1 al. (2002); Brummelkamp, T R. et al. (2002); U.S. Pat. Nos. 6,573,099 and 6,506,559; and International Patent Publication Nos. WO 01/36646, WO 99/32619, and WO 01/68836).

Ribozymes can also function as inhibitors of activin B or ALK3 receptor gene expression for use in the present invention. Ribozymes are enzymatic RNA molecules capable of catalyzing the specific cleavage of RNA. The mechanism of ribozyme action involves sequence specific hybridization of the ribozyme molecule to complementary target RNA, followed by endonucleolytic cleavage. Engineered hairpin or hammerhead motif ribozyme molecules that specifically and efficiently catalyze endonucleolytic cleavage of activin B or ALK3 receptor mRNA sequences are thereby useful within the scope of the present invention. Specific ribozyme cleavage sites within any potential RNA target are initially identified by scanning the target molecule for ribozyme cleavage sites, which typically include the following sequences, GUA, GUU, and GUC. Once identified, short RNA sequences of between about 15 and 20 ribonucleotides corresponding to the region of the target gene containing the cleavage site can be evaluated for predicted structural features, such as secondary structure, that can render the oligonucleotide sequence unsuitable. The suitability of candidate targets can also be evaluated by testing their accessibility to hybridization with complementary oligonucleotides, using, e.g., ribonuclease protection assays.

Both antisense oligonucleotides and ribozymes useful as inhibitors of activin B or ALK3 receptor gene expression can be prepared by known methods. These include techniques for chemical synthesis such as, e.g., by solid phase phosphoramadite chemical synthesis. Alternatively, anti-sense RNA molecules can be generated by in vitro or in vivo transcription of DNA sequences encoding the RNA molecule. Such DNA sequences can be incorporated into a wide variety of vectors that incorporate suitable RNA polymerase promoters such as the T7 or SP6 polymerase promoters. Various modifications to the oligonucleotides of the invention can be introduced as a means of increasing intracellular stability and half-life. Possible modifications include but are not limited to the addition of flanking sequences of ribonucleotides or deoxyribonucleotides to the 5′ and/or 3′ ends of the molecule, or the use of phosphorothioate or 2′-O-methyl rather than phosphodiesterase linkages within the oligonucleotide backbone.

Antisense oligonucleotides siRNAs and ribozymes of the invention may be delivered in vivo alone or in association with a vector. In its broadest sense, a “vector” is any vehicle capable of facilitating the transfer of the antisense oligonucleotide siRNA or ribozyme nucleic acid to the cells and preferably cells expressing activin B or ALK3. Preferably, the vector transports the nucleic acid to cells with reduced degradation relative to the extent of degradation that would result in the absence of the vector. In general, the vectors useful in the invention include, but are not limited to, plasmids, phagemids, viruses, other vehicles derived from viral or bacterial sources that have been manipulated by the insertion or incorporation of the antisense oligonucleotide siRNA or ribozyme nucleic acid sequences. Viral vectors are a preferred type of vector and include, but are not limited to nucleic acid sequences from the following viruses: retrovirus, such as moloney murine leukemia virus, harvey murine sarcoma virus, murine mammary tumor virus, and rouse sarcoma virus; adenovirus, adeno-associated virus; SV40-type viruses; polyoma viruses; Epstein-Barr viruses; papilloma viruses; herpes virus; vaccinia virus; polio virus; and RNA virus such as a retrovirus. One can readily employ other vectors not named but known to the art.

Preferred viral vectors are based on non-cytopathic eukaryotic viruses in which non-essential genes have been replaced with the gene of interest. Non-cytopathic viruses include retroviruses (e.g., lentivirus), the life cycle of which involves reverse transcription of genomic viral RNA into DNA with subsequent proviral integration into host cellular DNA. Retroviruses have been approved for human gene therapy trials. Most useful are those retroviruses that are replication-deficient (i.e., capable of directing synthesis of the desired proteins, but incapable of manufacturing an infectious particle). Such genetically altered retroviral expression vectors have general utility for the high-efficiency transduction of genes in vivo. Standard protocols for producing replication-deficient retroviruses (including the steps of incorporation of exogenous genetic material into a plasmid, transfection of a packaging cell lined with plasmid, production of recombinant retroviruses by the packaging cell line, collection of viral particles from tissue culture media, and infection of the target cells with viral particles) are provided in Kriegler, 1990 and in Murry, 1991).

Preferred viruses for certain applications are the adeno-viruses and adeno-associated viruses, which are double-stranded DNA viruses that have already been approved for human use in gene therapy. The adeno-associated virus can be engineered to be replication deficient and is capable of infecting a wide range of cell types and species. It further has advantages such as, heat and lipid solvent stability; high transduction frequencies in cells of diverse lineages, including hemopoietic cells; and lack of superinfection inhibition thus allowing multiple series of transductions. Reportedly, the adeno-associated virus can integrate into human cellular DNA in a site-specific manner, thereby minimizing the possibility of insertional mutagenesis and variability of inserted gene expression characteristic of retroviral infection. In addition, wild-type adeno-associated virus infections have been followed in tissue culture for greater than 100 passages in the absence of selective pressure, implying that the adeno-associated virus genomic integration is a relatively stable event. The adeno-associated virus can also function in an extrachromosomal fashion.

Other vectors include plasmid vectors. Plasmid vectors have been extensively described in the art and are well known to those of skill in the art. See e.g. Sambrook et al., 1989. In the last few years, plasmid vectors have been used as DNA vaccines for delivering antigen-encoding genes to cells in vivo. They are particularly advantageous for this because they do not have the same safety concerns as with many of the viral vectors. These plasmids, however, having a promoter compatible with the host cell, can express a peptide from a gene operatively encoded within the plasmid. Some commonly used plasmids include pBR322, pUC18, pUC19, pRC/CMV, SV40, and pBlueScript. Other plasmids are well known to those of ordinary skill in the art. Additionally, plasmids may be custom designed using restriction enzymes and ligation reactions to remove and add specific fragments of DNA. Plasmids may be delivered by a variety of parenteral, mucosal and topical routes. For example, the DNA plasmid can be injected by intramuscular, eye, intradermal, subcutaneous, or other routes. It may also be administered by intranasal sprays or drops, rectal suppository and orally. It may also be administered into the epidermis or a mucosal surface using a gene-gun. The plasmids may be given in an aqueous solution, dried onto gold particles or in association with another DNA delivery system including but not limited to liposomes, dendrimers, cochleate and microencapsulation.

In a particular embodiment, the antisense oligonucleotide, siRNA, shRNA or ribozyme nucleic acid sequence is under the control of a heterologous regulatory region, e.g., a heterologous promoter. The promoter may be specific for Muller glial cells, microglia cells, endothelial cells, pericyte cells and astrocytes For example, a specific expression in Muller glial cells may be obtained through the promoter of the glutamine synthetase gene is suitable. The promoter can also be, e.g., a viral promoter, such as CMV promoter or any synthetic promoters.

Another object of the invention relates to a method for treating anemia of inflammation comprising administering to a subject in need thereof a therapeutically effective amount of compound which inhibits the binding of activin B to ALK3 receptor or a compound which is an inhibitor of activin B expression or ALK3 receptor expression as described above.

In one aspect, the invention relates to a method for treating anemia of inflammation comprising administering to a subject in need thereof a therapeutically effective amount of an inhibitor of the binding of activin B to ALK3 receptor as above described.

As used herein, the term “treating” or “treatment”, denotes reversing, alleviating, inhibiting the progress of, or preventing the disorder or condition to which such term applies, or reversing, alleviating, inhibiting the progress of, or preventing one or more symptoms of the disorder or condition to which such term applies.

In another embodiment, the anemia of inflammation is induced by a viral, bacterial, parasitic, or fungal infection, a malignancy, an auto-immune disorder (rheumatoid arthritis, systemic lupus erythematosus or connective tissue disease, vasculitis, sarcoidosis, inflammatory bowel disease), a chronic rejection after solid organ transplantation, or chronic kidney disease and inflammation.

### Pharmaceutical Composition

Another object of the invention relates to a therapeutic composition comprising a compound according to the invention for the treatment of anemia of inflammation.

Preferably, said compound is an inhibitor of the binding of activin B to ALK3 receptor or an inhibitor of activin B expression or ALK3 receptor expression.

Any therapeutic agent of the invention may be combined with pharmaceutically acceptable excipients, and optionally sustained-release matrices, such as biodegradable polymers, to form therapeutic compositions.

“Pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

The form of the pharmaceutical compositions, the route of administration, the dosage and the regimen naturally depend upon the condition to be treated, the severity of the illness, the age, weight, and sex of the patient, etc.

The pharmaceutical compositions of the invention can be formulated for a topical, oral, intranasal, parenteral, intraocular, intravenous, intramuscular or subcutaneous administration and the like.

Preferably, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The doses used for the administration can be adapted as a function of various parameters, and in particular as a function of the mode of administration used, of the relevant pathology, or alternatively of the desired duration of treatment.

In addition, other pharmaceutically acceptable forms include, e.g. tablets or other solids for oral administration; time release capsules; and any other form currently can be used.

Compounds of the invention may be administered in the form of a pharmaceutical composition, as defined below.

By a “therapeutically effective amount” is meant a sufficient amount of compound to treat and/or to prevent glaucoma disorder.

It will be understood that the total daily usage of the compounds and compositions of the present invention will be decided by the attending physician within the scope of sound medical judgment. The specific therapeutically effective dose level for any particular patient will depend upon a variety of factors including the disorder being treated and the severity of the disorder; activity of the specific compound employed; the specific composition employed, the age, body weight, general health, sex and diet of the patient; the time of administration, route of administration, and rate of excretion of the specific compound employed; the duration of the treatment; drugs used in combination or coincidental with the specific polypeptide employed; and like factors well known in the medical arts. For example, it is well within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Preferably, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the patient to be treated. A medicament typically contains from about 0.01 mg to about 500 mg of the active ingredient, preferably from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

### Diagnostic Method

The invention relates to a method for diagnosis of anemia of inflammation in a patient comprising a step consisting of detecting Activin B in a sample obtained from said patient.

In another embodiment, the anemia of inflammation is induced by a viral, bacterial, parasitic, or fungal infection, a malignancy, an auto-immune disorder (rheumatoid arthritis, systemic lupus erythematosus or connective tissue disease, vasculitis, sarcoidosis, inflammatory bowel disease), a chronic rejection after solid organ transplantation, or chronic kidney disease and inflammation.

Typically, the sample according to the invention may be a blood, plasma, serum, lymph or urine sample. In a particular embodiment, said sample is blood.

In a particular embodiment, one of the two subunit of the activin B, called βB activin subunit, is detected in a sample obtained from said patient for diagnosing anemia of inflammation.

The term “detecting” as used above includes qualitative and/or quantitative detection (measuring levels) with or without reference to a control. Typically, activin B expression may be measured for example by RT-PCR or immunohistochemistry performed on the sample.

Preferably, the invention relates to a method for diagnosis of anemia of inflammation in a patient comprising a step a) consisting of measuring activin B expression in a sample obtained from said patient. Preferably, the method of the invention further comprises a step of comparing activin B expression level obtained in step a) to a threshold level.

The “control” may be a healthy subject, i.e. a subject who does not suffer from any anemia of inflammation. The control may also be a subject suffering from anemia of inflammation. Preferably, said control is a healthy subject.

For example, detecting activin B expression in sample may be performed by measuring the expression level of activin B gene.

Typically, the detection comprises contacting the sample with selective reagents such as probes, primers or ligands, and thereby detecting the presence, or measuring the amount, of polypeptides or nucleic acids of interest originally present in the sample. Contacting may be performed in any suitable device, such as a plate, microtiter dish, test tube, well, glass, column. In specific embodiments, the contacting is performed on a substrate coated with the reagent, such as a nucleic acid array or a specific ligand array. The substrate may be a solid or semi-solid substrate such as any suitable support comprising glass, plastic, nylon, paper, metal, polymers and the like. The substrate may be of various forms and sizes, such as a slide, a membrane, a bead, a column, a gel, etc. The contacting may be made under any condition suitable for a detectable complex, such as a nucleic acid hybrid or an antibody-antigen complex, to be formed between the reagent and the nucleic acids or polypeptides of the sample.

In a particular embodiment, the expression level of activin B gene may be determined by determining the quantity of mRNA of activin B gene. Such method may be suitable to measure the expression level of activin B gene in the sample.

Methods for measuring the quantity of mRNA are well known in the art. For example the nucleic acid contained in the samples (e.g., cell or tissue prepared from the patient) is first extracted according to standard methods, for example using lytic enzymes or chemical solutions or extracted by nucleic-acid-binding resins following the manufacturer's instructions. The extracted mRNA may be then detected by hybridization (e. g., Northern blot analysis).

Alternatively, the extracted mRNA may be subjected to coupled reverse transcription and amplification, such as reverse transcription and amplification by polymerase chain reaction (RT-PCR), using specific oligonucleotide primers that enable amplification of a region in activin B gene. Preferably quantitative or semi-quantitative RT-PCR is used. Real-time quantitative or semi-quantitative RT-PCR is particularly advantageous. Extracted mRNA may be reverse-transcribed and amplified, after which amplified sequences may be detected by hybridization with a suitable probe or by direct sequencing, or any other appropriate method known in the art.

Other methods of amplification include ligase chain reaction (LCR), transcription-mediated amplification (TMA), strand displacement amplification (SDA) and nucleic acid sequence based amplification (NASBA).

Nucleic acids having at least 10 nucleotides and exhibiting sequence complementarity or homology to the mRNA of interest herein find utility as hybridization probes or amplification primers. It is understood that such nucleic acids need not be identical, but are typically at least about 80% identical to the homologous region of comparable size, more preferably at least 85% identical and even more preferably at least 90%, preferably at least 95% identical. In certain embodiments, it will be advantageous to use nucleic acids in combination with appropriate means, such as a detectable label, for detecting hybridization. A wide variety of appropriate indicators are known in the art including, fluorescent, radioactive, enzymatic or other ligands (e. g. avidin/biotin).

Probes typically comprise single-stranded nucleic acids of between 10 to 1000 nucleotides in length, for instance of between 10 and 800, more preferably of between 15 and 700, typically of between 20 and 500. Primers typically are shorter single-stranded nucleic acids, of between 10 to 25 nucleotides in length, designed to perfectly or almost perfectly match a nucleic acid of interest, to be amplified. The probes and primers are “specific” to the nucleic acids they hybridize to, i.e. they preferably hybridize under high stringency hybridization conditions (corresponding to the highest melting temperature Tm, e.g., 50% formamide, 5× or 6×SCC. SCC is a 0.15 M NaCl, 0.015 M Na-citrate).

In a particular embodiment, the method of the invention comprises the steps of providing total RNAs obtained from the sample of the patient, and subjecting the RNAs to amplification and hybridization to specific probes, more particularly by means of a quantitative or semi-quantitative RT-PCR.

Total RNAs can be easily extracted from the sample. For instance, the sample may be treated prior to its use, e.g. in order to render nucleic acids available. Techniques of cell or protein lysis, concentration or dilution of nucleic acids, are known by the skilled person.

In another embodiment, the expression level of activin B gene may be measured by DNA microarray analysis. Such DNA microarray or nucleic acid microarray consists of different nucleic acid probes that are chemically attached to a substrate, which can be a microchip, a glass slide or a microsphere-sized bead. A microchip may be constituted of polymers, plastics, resins, polysaccharides, silica or silica-based materials, carbon, metals, inorganic glasses, or nitrocellulose. Probes comprise nucleic acids such as cDNAs or oligonucleotides that may be about 10 to about 60 base pairs. To measure the expression level of activin B gene, a sample from a test subject, optionally first subjected to a reverse transcription, is labelled and contacted with the microarray in hybridization conditions, leading to the formation of complexes between target nucleic acids that are complementary to probe sequences attached to the microarray surface. The labelled hybridized complexes are then detected and can be quantified or semi-quantified. Labelling may be achieved by various methods, e.g. by using radioactive or fluorescent labelling. Many variants of the microarray hybridization technology are available to the man skilled in the art (see e.g. the review by Hoheisel, Nature Reviews, Genetics, 2006, 7:200-210).

Detection of activin B expression in the sample may also be performed by measuring the level of activin B protein. In the present application, the “level of activin B protein” means the quantity or concentration of said activin B protein.

Such methods comprise contacting a sample with a binding partner capable of selectively interacting with activin B protein present in the sample. The binding partner is generally an antibody that may be polyclonal or monoclonal, preferably monoclonal.

The presence of the protein can be detected using standard electrophoretic and immunodiagnostic techniques, including immunoassays such as competition, direct reaction, or sandwich type assays. Such assays include, but are not limited to, Western blots; agglutination tests; enzyme-labeled and mediated immunoassays, such as ELISAs; biotin/avidin type assays; radioimmunoassays; immunoelectrophoresis; immunoprecipitation, etc. The reactions generally include revealing labels such as fluorescent, chemiluminescent, radioactive, enzymatic labels or dye molecules, or other methods for detecting the formation of a complex between the antigen and the antibody or antibodies reacted therewith. More preferably, determination of the concentrations of cholesterol and lactate are performed with a fluorescence-activated cell sorter (FACS). Said fluorescence-activated cell sorter is a machine that can rapidly separate the cells in a suspension on the basis of size and the color of their fluorescence.

The aforementioned assays generally involve separation of unbound protein in a liquid phase from a solid phase support to which antigen-antibody complexes are bound. Solid supports which can be used in the practice of the invention include substrates such as nitrocellulose (e. g., in membrane or microtiter well form); polyvinylchloride (e. g., sheets or microtiter wells); polystyrene latex (e.g., beads or microtiter plates); polyvinylidine fluoride; diazotized paper; nylon membranes; activated beads, magnetically responsive beads, and the like.

More particularly, an ELISA method can be used, wherein the wells of a microtiter plate are coated with a set of antibodies against the proteins to be tested. A sample containing or suspected of containing the marker protein is then added to the coated wells. After a period of incubation sufficient to allow the formation of antibody-antigen complexes, the plate(s) can be washed to remove unbound moieties and a detectably labeled secondary binding molecule is added. The secondary binding molecule is allowed to react with any captured sample marker protein, the plate is washed and the presence of the secondary binding molecule is detected using methods well known in the art.

Various immunoenzymatic staining methods are known in the art for detecting a protein of interest. For example, immunoenzymatic interactions can be visualized using different enzymes such as peroxidase, alkaline phosphatase, or different chromogens such as DAB, AEC, or Fast Red; or fluorescent labels such as FITC, Cy3, Cy5, Cy7, Alexafluors, etc. Counterstains may include H&E, DAPI, Hoechst, so long as such stains are compatable with other detection reagents and the visualization strategy used. As known in the art, amplification reagents may be used to intensify staining signal. For example, tyramide reagents may be used. The staining methods of the present invention may be accomplished using any suitable method or system as would be apparent to one of skill in the art, including automated, semi-automated or manual systems.

The method of the invention may comprise a further step consisting of comparing activin B expression with a control reference.

The invention thus relates to a method for diagnosis anemia of inflammation in a patient comprising determining the expression level of activin B in a sample obtained from said patient and comparing said expression level to a threshold value. As used herein, “expression level of activin B” refers to an amount or a concentration of a transcription product, for instance mRNA coding for activin B, or of a translation product, for instance the protein activin B. Typically, a level of mRNA expression can be expressed in units such as transcripts per cell or nanograms per microgram of tissue. A level of a polypeptide can be expressed as nanograms per microgram of tissue or nanograms per milliliter of a culture medium, for example. Alternatively, relative units can be employed to describe an expression level.

In a particular embodiment, when the measure of activin B gene expression is performed by rt qPCR, the expression level of activin B gene in a patient suffering of anemia of inflammation is increased by at least 30%, preferably by at least 40%, preferably by at least 50%; preferably by at least 60%, preferably by at least 70%, preferably by at least 80%, more preferably by at least 90%, even more at least 100% compared to a control reference. In other words, preferably, when activin B gene expression is measured by rt qPCR, the quantity of mRNA encoding activin B gene in a patient suffering of anemia of inflammation is increased by at least 30%, preferably by at least 40%, preferably by at least 50%; preferably by at least 60%, preferably by at least 70%, preferably by at least 80%, more preferably by at least 90%, even more at least 100% or more than 100% compared to a control reference.

In a particular embodiment, a threshold value may be established to easily diagnose anemia of inflammation.

Typically, a “threshold value”, “threshold level” or “cut-off value” can be determined experimentally, empirically, or theoretically. A threshold value can also be arbitrarily selected based upon the existing experimental and/or clinical conditions, as would be recognized by a person of ordinary skilled in the art. Preferably, the person skilled in the art may compare the expression levels of activin B obtained according to the method of the invention with a defined threshold value.

Preferably, said threshold value is the mean expression level of activin B of a population of healthy individuals. As used herein, the term “healthy individual” denotes a human which is known to be healthy, i.e. which does not suffer from anemia of inflammation, has never been subjected to such anemia of inflammation, and does not need any medical care.

Typically, the skilled person in the art may determine the expression level of activin B in a biological sample, preferably blood, of 100 individuals known to be healthy. The mean value of the obtained expression levels is then determined, according to well known statistical analysis, so as to obtain the mean expression level of activin B. Said value is then considered as being normal and thus constitute a threshold value. By comparing the expression levels of activin B to this threshold value, the physician is then able to diagnose anemia of inflammation. Indeed, by comparing the expression level of activin B obtained in a biological sample, preferably blood, of a given subject to a threshold value, one can easily determine whether said subject suffers from anemia of inflammation or not.

Accordingly, the physician would be able to adapt and optimize appropriate medical care of a subject in a critical and life-threatening condition suffering from anemia of inflammation. The determination of said diagnostic is highly appropriate for follow-up care and clinical decision making.

Therefore, the invention is drawn to a method for diagnosis of anemia of inflammation in a patient comprising the following steps:

a) determining the level of expression of activin B in a sample obtained from said patient;

b) determining the mean expression level of activin B in a biological sample of a population of healthy individuals, preferably 100 healthy individuals; and

c) a step of comparing the expression level of activin B obtained of a) to the mean expression level of activin B obtained in b).

The present invention also relates to kits for the diagnosis of anemia of inflammation, comprising means for detecting activin B expression.

According to the invention, the kits of the invention may comprise an anti-activin B protein antibody; and another molecule coupled with a signalling system which binds to said activin B protein antibody.

Typically, the antibodies or combination of antibodies are in the form of solutions ready for use. In one embodiment, the kit comprises containers with the solutions ready for use. Any other forms are encompassed by the present invention and the man skilled in the art can routinely adapt the form to the use in immunohistochemistry.

The present invention also relates to activin B gene or protein as a biomarker for the diagnosis of anemia of inflammation.

In another embodiment, the invention relates to an in vitro method for monitoring a patient's response to anemia of inflammation treatment which comprises a step of measuring the expression level of activin B gene, or a step of measuring the level of activin B protein, in a sample from a patient.

Thus, the present invention provides for the use of activin B gene or protein as a biomarker for the monitoring of anti anemia of inflammation therapies.

According to the invention, the expression level of activin B gene or the level of activin B protein may be determined to monitor a patient's response to anemia of inflammation treatment.

The invention will be further illustrated by the following figures and examples. However, these examples and figures should not be interpreted in any way as limiting the scope of the present invention.

### FIGURES

FIG. 1. Inflammation upregulates hepcidin expression via phosphorylation of the Smad effectors 1, 5, and 8, and independently of Bmp6. Groups of 6 mice (3 wild-type and 3. Bmp6−/−) were sacrificed at different time points following administration of LPS (1 μg/g body weight). (A) Hepcidin (Hamp) mRNA levels were measured by qRT-PCR. Values shown are means of −ΔCt (i.e., Ct Hprt−Ct Hamp)±SD. Four hours after LPS administration, hepcidin mRNA levels were increased about 2.5-fold (−ΔΔCt=1.31±0.54) in wild-type mice and 40-fold (−ΔΔCt=5.37±1.67) in Bmp6−/− mice. Means of −ΔCt values between LPS-challenged and unchallenged mice of each genotype were compared by Student's t-tests (***, p<0.001; **, p<0.01; *, p<0.05). (B) Protein extracts were prepared from the livers of 6 mice at each time point. Phospho-Stat3, total Stat3, phospho-Smad1/5/8 and total Smad5 were detected by immunoblot techniques in wild-type (left) and Bmp6−/− (right) mice. The blots shown are representative of three independent experiments for each time point and each mouse genotype.

FIG. 2. Inflammation dramatically increases transcription of activin B, but down-regulates that of activin A, even in the absence of Bmp6. Groups of 6 mice (3 wild-type and 3 Bmp6−/−) were sacrificed at different time points following administration of LPS (1 μg/g body weight). mRNA levels of Inhbb, coding for the βB activin subunit (A), and Inhba, coding for the βA activin subunit (B), were measured by qRT-PCR. Values shown are means of −ΔCt (i.e., Ct Hprt−Ct target gene)±SD. Four hours after LPS administration, Inhbb mRNA levels were increased about 35-fold (−ΔΔCt=5.14±0.39) in wild-type mice and 45-fold (−ΔΔCt=5.58±1.22) in Bmp6−/− mice. Effect of LPS on target Inhbb or Inhba gene expression independently of the mouse genotype was assessed by two-way ANOVA (***, p<0.001; **, p<0.01).

FIG. 3. Inflammation upregulates activin B and hepcidin transcription in the absence of 11-6. Groups of 4 Il-6−/− mice were injected with LPS (1 μg/g body weight) or saline and were sacrificed 4 hours later. (A) mRNA levels of Inhbb and Hamp were measured by qRT-PCR. Values shown are means of −ΔCt (i.e., Ct Hprt−Ct targe genet)±SD. Values obtained for Bmp6−/− mice injected with LPS or saline and examined 4 hours later are provided for comparison. Effect of LPS on Inhbb and Hamp gene expression was assessed by Student's t-test (***, p<0.001). (B) Protein extracts were prepared from the mouse livers of the Il-6−/− mice injected with LPS or saline. Phospho-Stat3, total Stat3, phospho-Smad1/5/8 and total Smad5 were detected by immunoblot techniques.

FIG. 4. Activin B induces hepcidin expression and SMAD1/5/8 phosphorylation in human hepatoma-derived cells. HepG2 cells were treated with IL-6 (50 ng/ml) and/or activin B (50 ng/ml) or activin A (50 ng/ml) for 1 h30, 2 h30, 4 h, 6 h, and 24 h. (A) Hepcidin (Hamp) mRNA levels were measured by qRT-PCR. Values shown are means of −ΔΔCt (i.e., −ΔCt treatment+ΔCt vehicle)±SD obtained from four independent experiments. Means of −ΔΔCt were compared to 0 by Student's t-tests (***, p<0.001; **, p<0.01; *, p<0.05). (B) Protein extracts were prepared from HepG2 cells treated with the different activators for 2 h30. Phospho-Smad1/5/8, total Smad5, phospho-Stat3 and total Stat3 were detected by immunoblot techniques. Similar patterns were observed after 4 and 6 hours of treatment (data not shown).

FIG. 5. Activin B uses the classical BMP type I receptors to regulate BMP signaling and hepcidin expression in human hepatoma-derived cells. HepG2 cells were pretreated with LDN-193189 (100 nM) or vehicule for 30 minutes and then stimulated with activin B (50 ng/ml) or vehicule for 2 h30, 4 h, or 6 h. (A) Hepcidin (Hamp) mRNA levels were measured by qRT-PCR. Values shown are −ΔΔCt (i.e., −ΔCt treatment+ΔCt vehicle)±SD obtained from three independent experiments. Means of −ΔΔCt were compared to 0 by Student's t-tests (**, p<0.01; *, p<0.05). (B) Protein extracts were prepared from these cells. Phospho-Smad1/5/8 and total Smad5 were detected by immunoblot techniques.

FIG. 6. Activin B uses the classical BMP type I receptors to induce SMAD1/5/8 phosphorylation and hepcidin expression in mouse primary hepatocytes. Mouse primary hepatocytes were treated with IL-6 (50 ng/ml) and/or activin B (50 ng/ml) for 2 h30. (A) Protein extracts were prepared from these hepatocytes. Phospho-Smad1/5/8, total Smad5, phospho-Stat3 and total Stat3 were detected by immunoblot techniques. The blot shown is representative of three independent experiments. (B) Hepcidin (Hamp) mRNA levels were measured by qRT-PCR. Values shown are means of −ΔΔCt (i.e., −ΔCt treatment+ΔCt vehicle)±SD obtained from four independent experiments. Means of −ΔΔCt were compared to 0 by Student's t-tests (**, p<0.01). (C) Mouse primary hepatocytes were treated with activin B (50 ng/ml) for 2 h30 in the presence or absence of LDN-193189 (1 μM). Phospho-Smad1/5/8 and total Smad5 were detected by immunoblot techniques. The blot shown is representative of three independent experiments.

FIG. 7. Inflammation increases transcription of Tnf and 11-6 similarly in the liver of wild-type mice and that of Bmp6-deficient mice. Groups of 6 mice (3 wild-type and 3 Bmp6−/−) were sacrificed at different time points following administration of LPS (1 μg/g body weight). mRNA levels of Tnf (A) and 11-6 (B) expressed in the liver were measured by qRT-PCR. Values shown are means of −ΔCt (i.e., Ct Hprt−Ct target gene)±SD. Effect of LPS on Tnf and IL-6 gene expression independently of the mouse genotype was assessed by two-way ANOVA (***, p<0.001).

FIG. 8. Inflammation increases transcription of Crp but decreases that of hemojuvelin similarly in the liver of wild-type mice and that of Bmp6-deficient mice. Groups of 6 mice (3 wild-type and 3 Bmp6−/−) were sacrificed at different time points following administration of LPS (1 μg/g body weight). mRNA levels of Crp (A) and Hjv (B) expressed in the liver were measured by qRT-PCR. Values shown are means of −ΔCt (i.e., Ct Hprt−Ct target gene)±SD. Effect of LPS on Crp and Hjv gene expression independently of the mouse genotype was assessed by two-way ANOVA (***, p<0.001; **, p<0.01).

FIG. 9. Inflammation down-regulates transcription of Bmp genes in the liver of both wild-type mice and Bmp6-deficient mice. Groups of 6 mice (3 wild-type and 3 Bmp6−/−) were sacrificed at different time points following administration of LPS (1 μg/g body weight). mRNA levels of Bmp2 (A), Bmp5 (B) and Bmp9 (C) expressed in the liver were measured by qRT-PCR. Values shown are means of −ΔCt (i.e., Ct Hprt−Ct target gene)±SD. Effect of LPS on the expression of the different Bmp genes independently of the mouse genotype was assessed by two-way ANOVA (***, p<0.001; **, p<0.01).

FIG. 10. Inflammation slightly reduces expression of Inhbc and more strongly down-regulates that if Inhbe in the liver of both wild-type mice and Bmp6-deficient mice. Groups of 6 mice (3 wild-type and 3 Bmp6−/−) were sacrificed at different time points following administration of LPS (1 μg/g body weight). mRNA levels of Inhbc, coding for the βC activin subunit (A), and Inhbe, coding for the βE activin subunit (B), were measured by qRT-PCR. Values shown are means of −ΔCt (i.e., Ct Hprt−Ct target gene)±SD. Effect of LPS on Inhbc and Inhbe gene expression independently of the mouse genotype was assessed by two-way ANOVA (***, p<0.001).

FIG. 11. Activin B uses ALK3 rather than ALK2 to regulate BMP signaling and hepcidin expression. HepG2 cells were pretreated with human recombinant ALK2 or ALK3 extracellular domains expressed as Fc fusion proteins (ALK2-Fc and ALK3-Fc) or vehicule for 30 minutes and then stimulated with activin B (5 ng/ml) for 2 h30. Hepcidin (Hamp) mRNA levels were measured by qRT-PCR. Values shown are means of −ΔΔCt (i.e., −ΔCt treatment+ΔCt vehicle)±SD obtained from five independent experiments. Means of −ΔΔCt were compared to 0 by Student's t-tests (**, p<0.01; *, p<0.05). In contrast to ALK2-Fc, ALK3-Fc prevents hepcidin upregulation by activin B.

### Example

### Material & Methods

Murine models.

To examine the effect of LPS (serotype 055:B5; Sigma) in the regulation of hepcidin, 7 to 8-week-old Bmp6 null mice (Bmp6m1Rob) and wild-type controls on a CD1 background, as well as 7-8-week-old B6.129S2-Il6tm1Kopf/J mice on a C57BL/6J background, were challenged with an intraperitoneal injection of LPS (1 μg/g body weight) or an equivalent volume of saline and livers were harvested at different time points (30 min, 1, 1 h30, 2, 4, 6, 15, 24 and 48 h) following injection. All experiments were performed on males and, unless otherwise specified, mice received a diet with standard iron content (250 mg iron/kg; SAFE, Augy, France). Experimental protocols were approved by the Midi-Pyrénées Animal Ethics Committee.

HepG2 Cells.

Human hepatoma cells (HepG2; ATCC) were cultured in high glucose Dulbecco's Modified Eagle Media (DMEM) supplemented with 10% fetal calf serum (FCS) and antibiotics. For stimulation experiments, cells were transferred to 6-well dishes and starved with DMEM containing 0.1% FCS for 5 hours before exposure to IL-6 (50 ng/ml; R&D Systems) and/or activin A (50 ng/ml; R&D) or activin B (50 ng/ml; R&D) for 1 h30, 2 h30, 4 h, 6 h, and 24 h. The pharmacological inhibitor LDN-193189 (100 nM; Axon Medchem), ALK2-Fc (1 μg/mL; R&D), or ALK3-Fc (1 μg/mL; R&D) were added 30 min prior to activin B exposure.

Mouse Primary Hepatocyte Cultures.

Mouse primary hepatocytes were isolated from ten-week-old Bmp6-deficient mice using a collagenase perfusion protocol (Lin et al., 2007). The viable hepatocyte population was further purified by a Percoll gradient centrifugation. Hepatocytes were plated in 6-well collagen-coated plates at 7×105 cells per well and cultured in DMEM supplemented with 10% FCS and antibiotics for 18 hours. After serum starvation for 5 hours in 0.1% FCS, hepatocytes were stimulated with IL-6 (50 ng/ml) and/or activin B (50 ng/ml), or vehicle, in the presence or absence of LDN-193189 (l μM), and RNA was harvested 1, 2, 3, and 4 hours later.

Quantitation of mRNA Levels.

Total RNA from mouse liver, human hepatoma cells, or mouse primary hepatocytes, was extracted using Trizol (Invitrogen). cDNA was synthesized using MMLV-RT (Promega). Quantitative PCR (Q-PCR) reactions were prepared with LightCycler® 480 DNA SYBR Green I Master reaction mix (Roche Diagnostics) and run in duplicate on a LightCycler® 480 Instrument (Roche Diagnostics).

Protein Extraction.

Livers were homogenized in a FastPrep®-24 Instrument (MP Biomedicals) for 15 sec at 4 m/s. The lysis buffer (50 mM Tris-HCl, pH 8, 150 mM NaCl, 5 mM EDTA, pH 8, 0.1% NP-40) included inhibitors of proteases (complete protease inhibitor cocktail, Roche Applied Science) and of phosphatases (phosphatase inhibitor cocktail 2, Sigma-Aldrich). Liver proteins were quantified using the Bio-Rad Protein Assay kit. HepG2 cells and mouse primary hepatocytes were lysed in RIPA buffer (Sigma-Aldrich) supplemented with protease and phosphatase inhibitors, and proteins were quantified using the Bio-Rad DC Protein Assay.

Western Blot Analysis.

Fresh protein extracts were diluted in Laemmli buffer (Sigma-Aldrich), incubated for 5 minutes at 95° C., and subjected to SDS-PAGE. Proteins were then transferred to PVDF membranes (Millipore). Membranes were blocked with 5% of dry milk in TBS-T buffer (10 mM Tris-HCl, pH 7.5, 150 mM NaCl, 0.15% Tween 20). They were incubated with rabbit antibodies to phospho-Smad1/5/8 (Cell Signaling Technology) or phospho-Stat3 (Cell Signaling) at 4° C. overnight, and washed with TBS-T buffer. Following incubation with a goat anti-rabbit IgG antibody (Cell Signaling) conjugated to horseradish peroxidase (HRP), enzyme activity was visualized by an ECL-based detection system (Millipore). Blots were then stripped and reprobed with rabbit antibodies to Smad5 (Epitomics) or Stat3 (Cell Signaling) 2 hours at RT before incubation with the goat anti-rabbit HRP-linked antibody.

Statistical Analyses.

Means of −ΔCt (i.e., Ct Hprt−Ct target)±standard deviation (SD) were calculated for the different groups of mice. Means of −ΔCt values between LPS-challenged and unchallenged mice were compared by Student's t-tests. Because, in addition to inflammation, mouse genotype (wild-type versus Bmp6−/−) may also affect mean −ΔCt values, we used two-way analysis of variance (ANOVA) to test for the effect of both factors and their interaction. Differences between mean −ΔCt at a specific time point after LPS administration and mean −ΔCt at time 0 (−ΔΔCt±SD) were also calculated and point estimations of fold changes induced by LPS administration obtained by raising 2 to the power −ΔΔCt (Yuan et al., 2006). For HepG2 cells or mouse primary hepatocytes, −ΔΔCt values (i.e., −ΔCt treatment+ΔCt vehicle)±SD were obtained for each condition and means of −ΔΔCt were compared to 0 by Student's t-tests.

Results

Inflammation Upregulates Hepcidin Independently of BMP6.

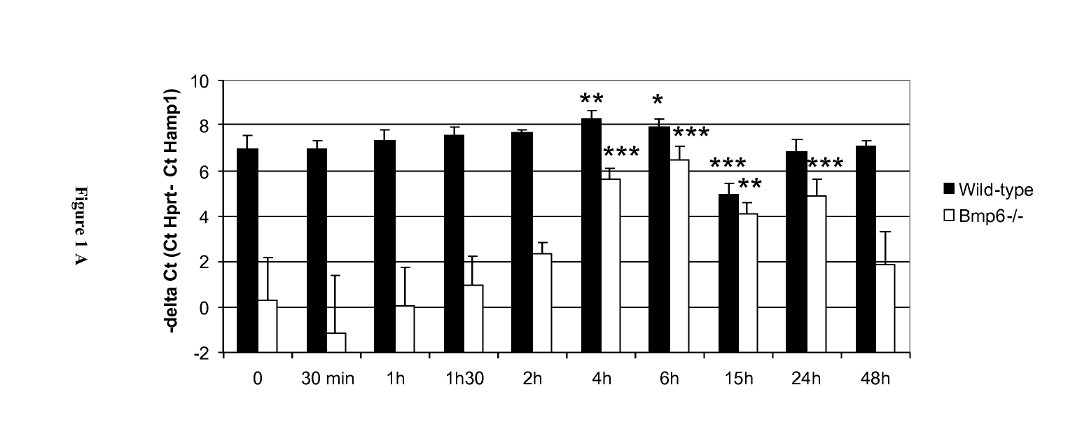

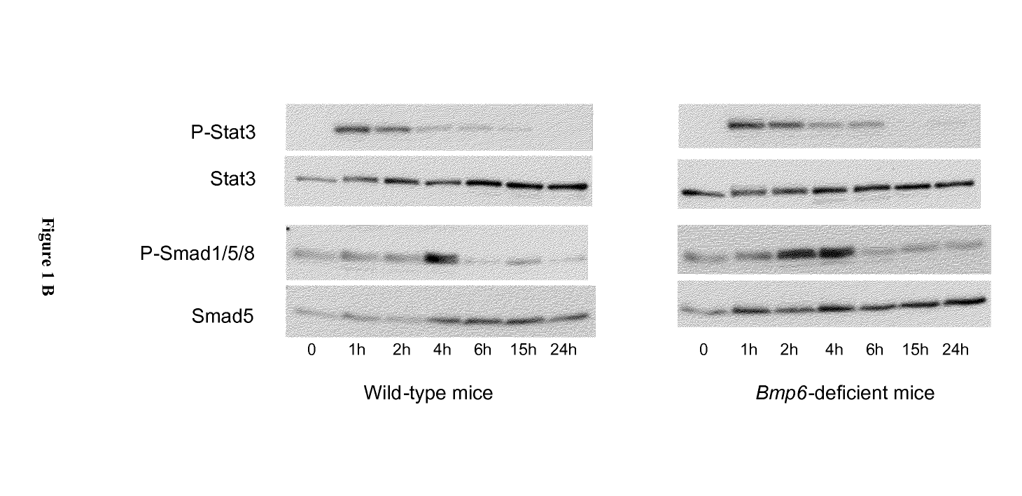

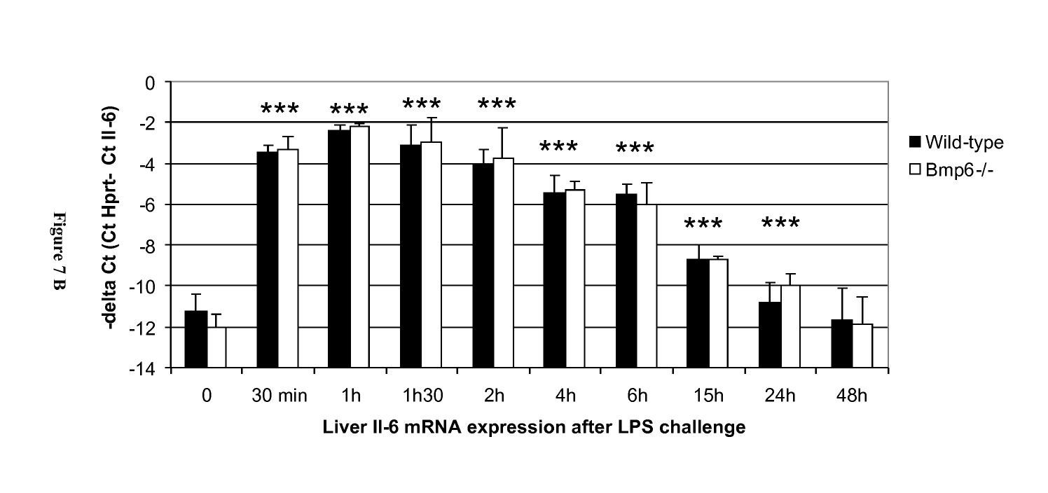

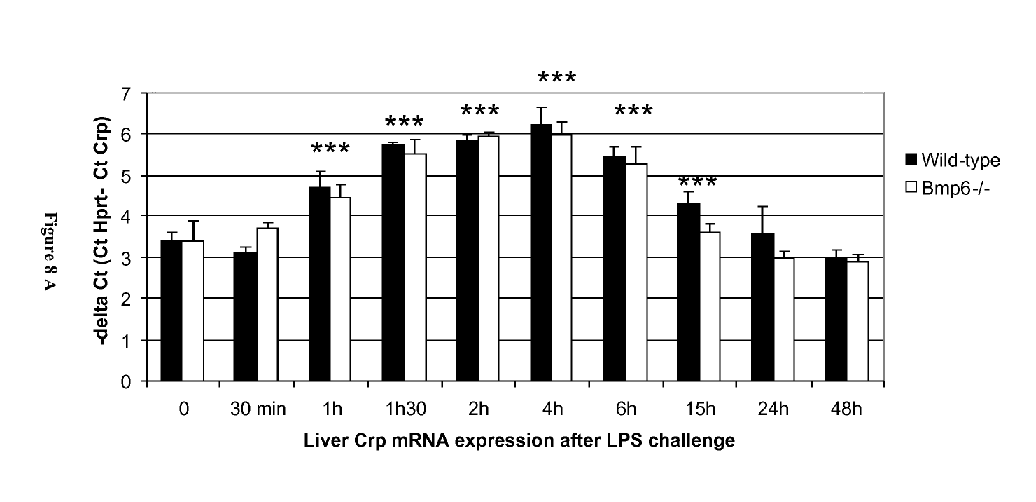

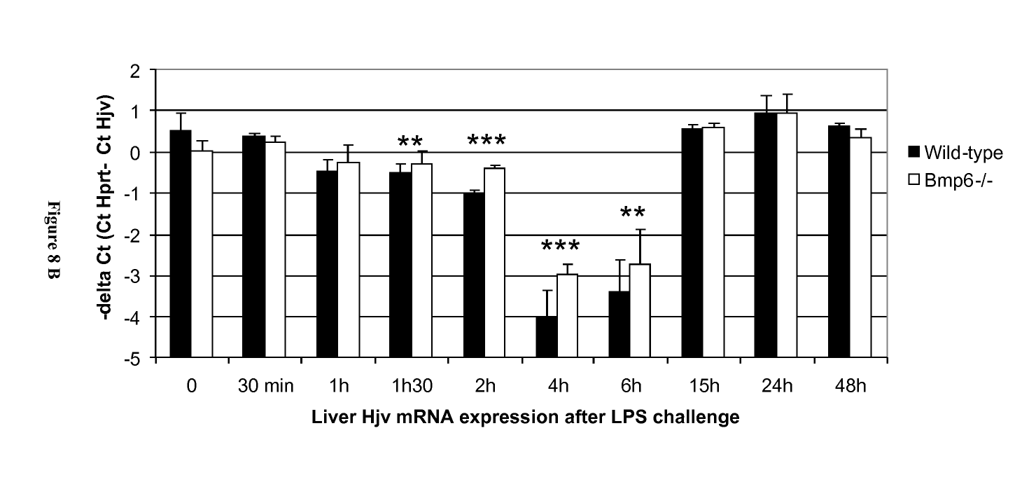

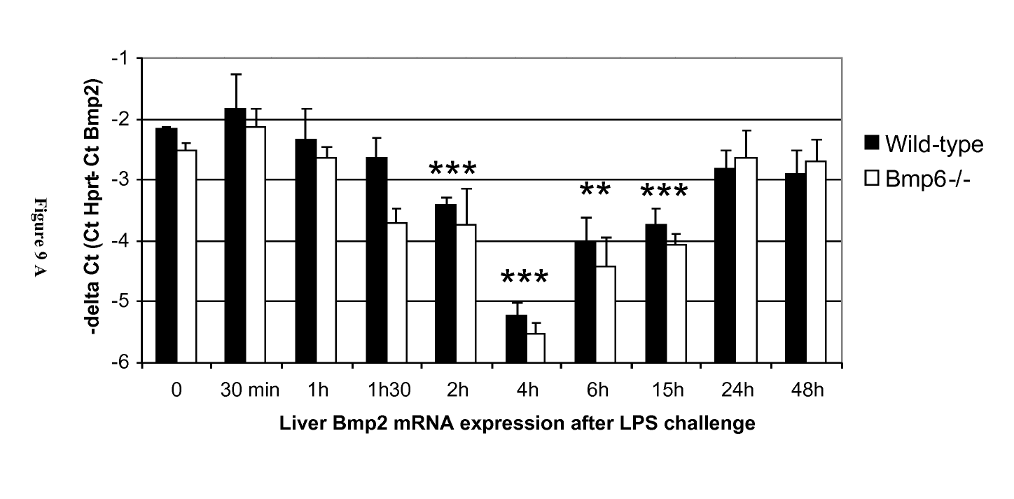

To investigate the detailed timing of the inflammatory response induced by the Gram-negative bacterial cell wall component, groups of six mice on a CD1 background (3 wild-type and 3 Bmp6−/−) were sacrificed at different time points (30 min, 1 h, 1 h30, 2, 4, 6, 15, 24 and 48 h) following challenge with LPS. One group of six CD1 mice (3 wild-type and 3 Bmp6−/−) was killed to provide baseline values. As expected, LPS induced a rapid and massive elevation of Tnf and 11-6 mRNA expression in the liver of both wild-type and Bmp6−/− mice (FIG. 7). Noteworthy, the magnitude of this induction was similar in the two groups of mice. This was followed by an increase in the liver acute-phase protein Crp mRNA (FIG. 8A). As shown on FIG. 1A, wild-type mice progressively increased their hepcidin (Hamp) mRNA up to a maximum of 2.5-fold their initial value (−ΔΔCt=1.34±0.54) 4 hours after LPS administration. Because Bmp6−/− mice have very low basal hepcidin levels, this increase was even more significant (over 40 fold at 4 h; −ΔΔCt=5.37±1.67) in Bmp6−/− than in wild-type mice. This confirms that Bmp6 is not necessary for the induction of hepcidin by LPS. In contrast to Hamp−/− or iron-deprived mice (De Domenico et al.; Pagani et al.), Bmp6−/− mice do not have a proinflammatory condition exacerbated by LPS when compared with wild-type mice (FIG. 7), probably because their capacity to upregulate hepcidin in the inflammatory context is preserved. Interestingly, hemojuvelin (Hjv) mRNA was strongly down-regulated (over 8-fold) in the liver of both wild-type and Bmp6−/− mice at the time points where hepcidin levels were maximum, i.e. between 4 and 6 hours after LPS administration (FIG. 8B).

The LPS challenge increases phosphorylation of Smad1/5/8 effectors in the liver of both wild-type and Bmp6−/− mice.

As expected, LPS rapidly stimulated Stat3 activation. Phosphorylation of Stat3 reached a peak 1 hour after LPS administration, i.e. 3 hours ahead of hepcidin maximal induction, and then returned gradually to basal levels (FIG. 1B). Because pharmacologic inhibition of BMP type I receptors blocks induction of hepcidin expression by various inflammatory stimuli, we also checked whether LPS was able to induce BMP/Smad signaling even in the absence of Bmp6. As shown on FIG. 1B, phosphorylation of the Smad1/5/8 effectors was significantly increased 2 to 4 hours after LPS challenge, i.e. concurrently with the increase in Hamp mRNA expression, in the liver of both wild-type and Bmp6−/− mice, and returned to basal levels at 6 h. This provides evidence supporting a role for BMP/Smad signaling in the induction of hepcidin by LPS and suggests that a ligand of the TGF-β/BMP superfamily, other than BMP6, activates hepcidin production during inflammation.

None of the ligands of the BMP sub-family has its expression upregulated following LPS administration.

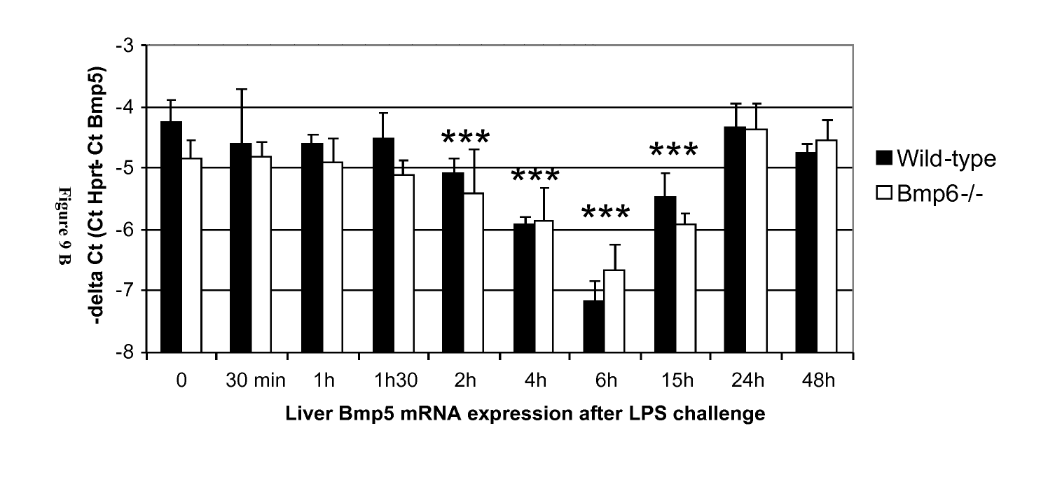

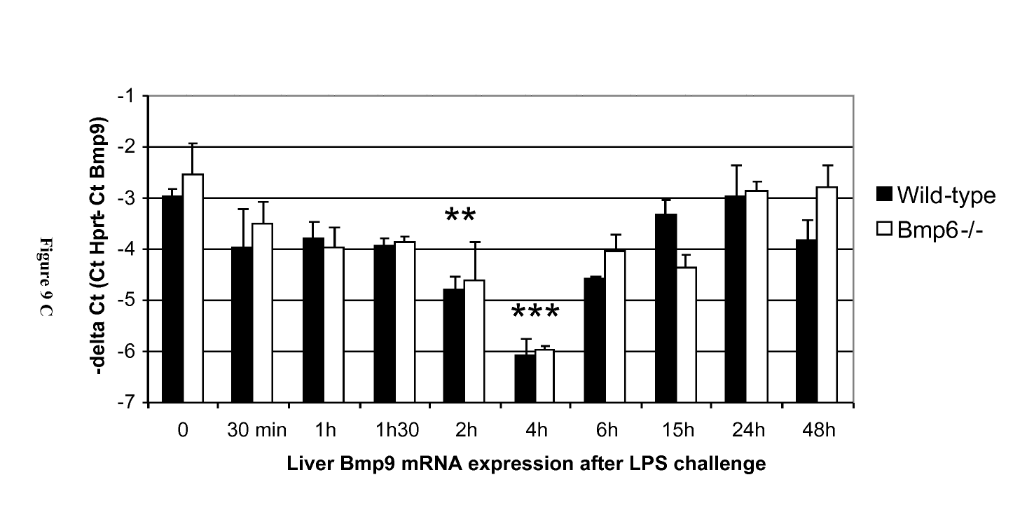

Because Bmp6 is not necessary for induction of hepcidin by inflammation, we suspected that another ligand of the Bmp sub-family was involved in this process and that its expression was induced by inflammation as Bmp6 expression was induced by iron. We therefore compared liver mRNA expression of Bmp2, Bmp4, Bmp5, Bmp6 (in wild-type mice), Bmp7, and Bmp9 between LPS-challenged and control mice. All these Bmp molecules had previously been shown to robustly increase hepcidin mRNA expression in hepatoma-derived cells. Rather than being increased by LPS, expression of these Bmp ligands was either unchanged or even significantly decreased 2 to 15 hours after LPS administration, as shown for Bmp2, Bmp5, and Bmp9 on FIG. 9.

In contrast to Bmp mRNAs, activin B expression is highly upregulated by inflammation in the liver of both wild-type and Bmp6−/− mice.

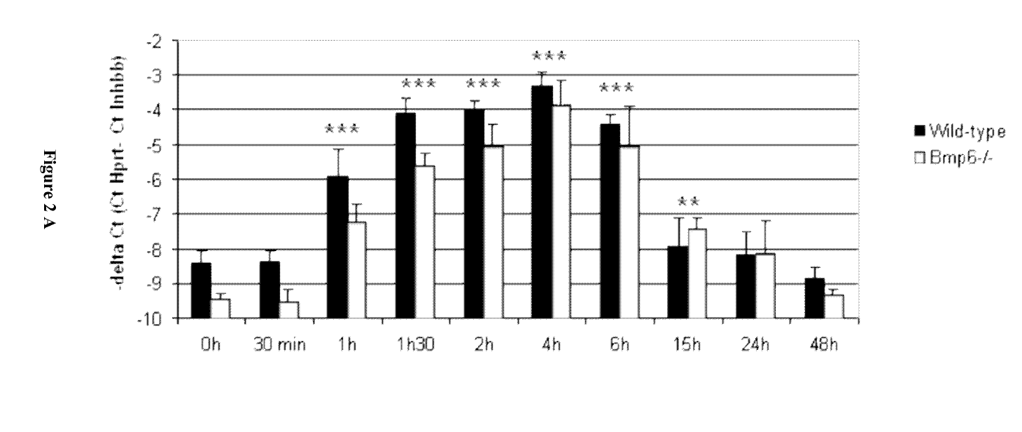

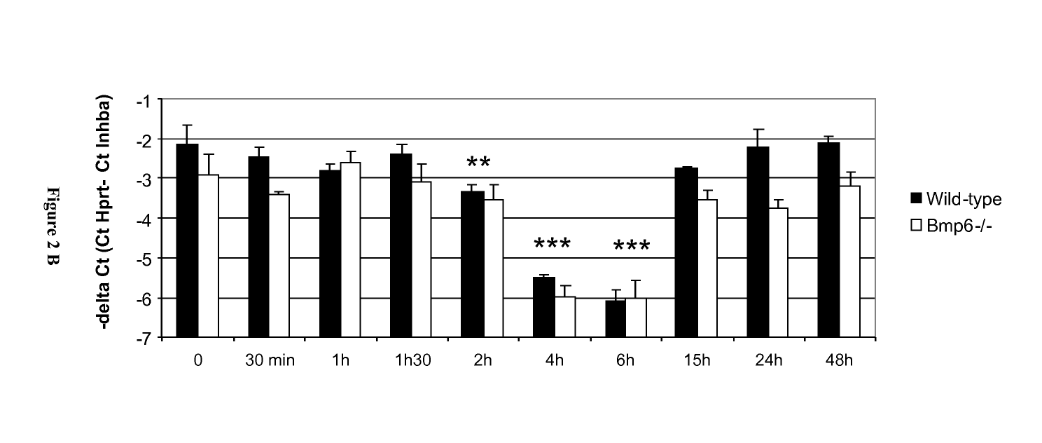

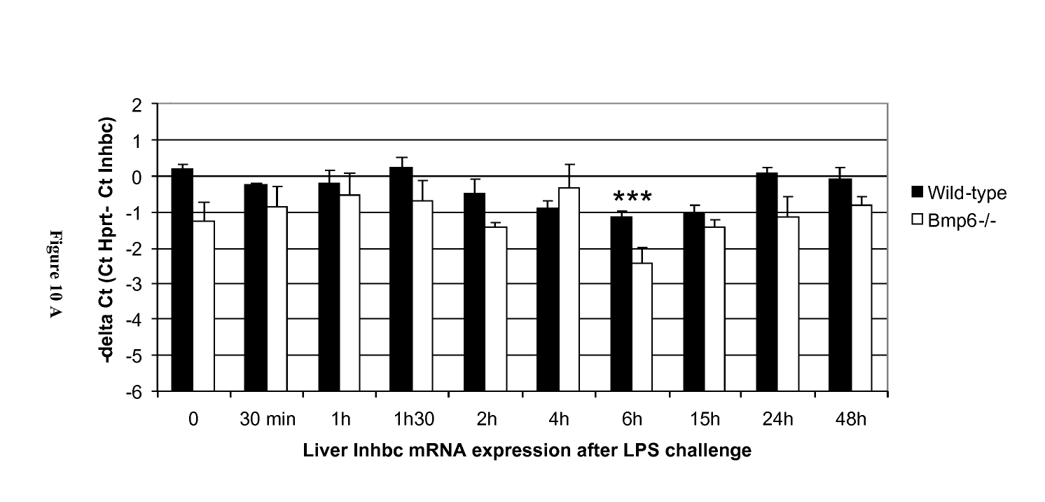

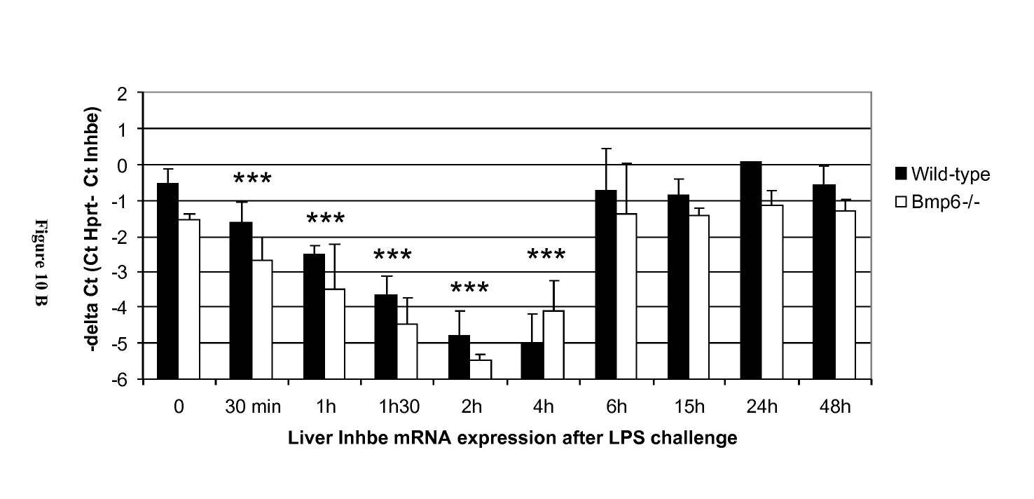

In hepatoma-derived cell lines, endogenous ligands BMP2, BMP4, and BMP6 have been shown to signal through the BMP type I receptors ALK3 and/or ALK2 to regulate hepcidin expression, a process facilitated by hemojuvelin. Interestingly, the expression of other members of the TGF-β superfamily, activin A and activin B, is increased in inflammatory diseases such as septicemia, inflammatory bowel disease and rheumatoid arthritis, which raises the possibility that activins play a significant role in the acute inflammatory response. We therefore hypothesized that, although activins conventionally use their own receptors, ALK4 or ALK7, they could under some circumstances bind type I receptors used by BMP ligands and activate hepcidin through phosphorylation of the Smad effectors 1, 5, and 8. We then tested whether the mRNA expression of Inhba, Inhbb, Inhbc and/or Inhbe, encoding the βA, βB, βC, and βE activin subunits, respectively, was induced by LPS. As shown on FIG. 2A, there was a dramatic increase in Inhbb mRNA starting 1 h after LPS administration and peaking at 4 h, with a 35-fold increase (−ΔΔCt=5.14±0.39) in wild-type mice and a 45-fold increase (−ΔΔCt=5.58±1.22) in Bmp6−/− mice. In contrast, Inhba mRNA undergoes a profound decrease (about 10-fold in wild-type and 8-fold in Bmp6−/− mice) between 4 and 6 hours after the LPS challenge (FIG. 2B). Expression of Inhbc mRNA was only modestly affected by LPS (FIG. 10A) but that of Inhbe was strongly reduced up to 4 hours after LPS administration (FIG. 10B). These findings show that, among all activin molecules, activin B is the only candidate ligand whose expression increases after an LPS challenge.

The upregulation of activin B transcription is independent of IL-6.

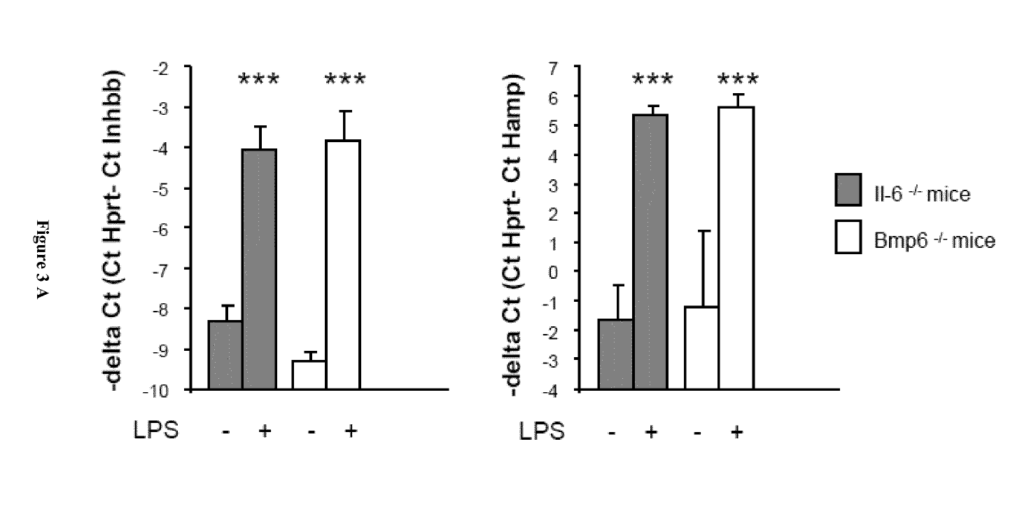

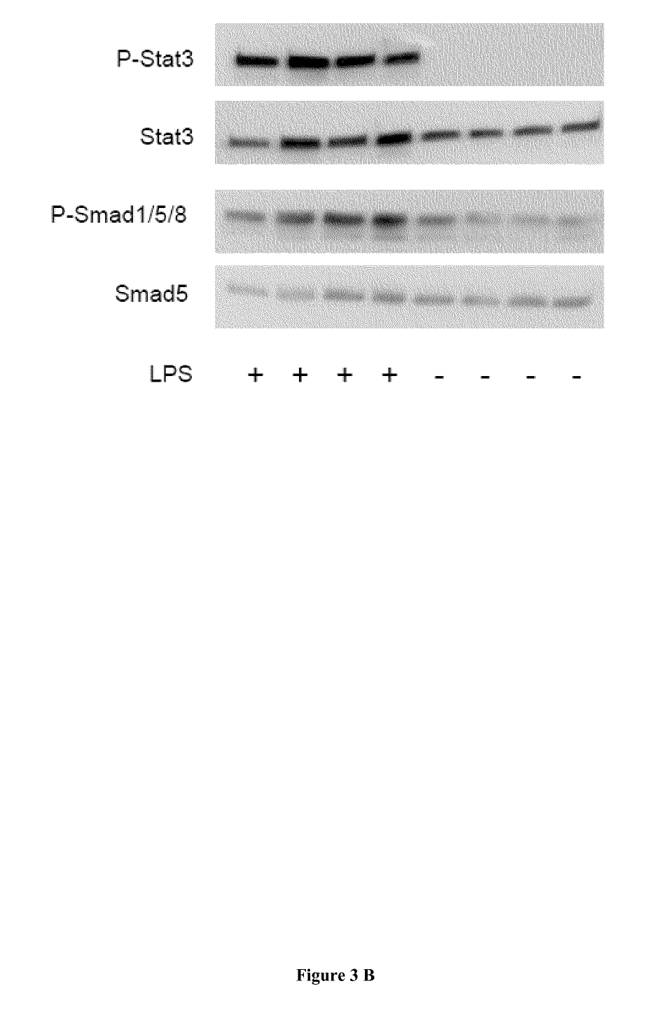

Because IL-6 is sufficient for the induction of hepcidin during inflammation (Nemeth et al., 2004a), we next examined whether IL-6−/− mice were able to increase activin B transcription in response to an LPS challenge. As previously done, Il-6−/− mice of the C57BL/6 background were switched to an iron-deficient diet containing 2-4 ppm iron 12 days prior to the experiment. They were killed 4 hours after LPS administration and their activin B and hepcidin levels were compared to those of unchallenged Il-6−/− mice. Interestingly, as shown on FIG. 3A, Il-6−/− mice were able to upregulate both Inhbb and Hamp mRNA in response to LPS to a level similar to that of LPS-challenged Bmp6−/− mice. These data show that induction of activin B and hepcidin by LPS occurs independently of 11-6 and despite the switch-off of the iron sensing pathway obtained either by feeding the mice an iron-deficient diet or in the absence of the key ligand Bmp6. As for the wild-type and Bmp6−/− mice studied above, Inhba and Hjv mRNA expression was strongly reduced in the liver of Il-6−/− mice 4 hours after LPS administration (data not shown), whereas phosphorylation of both Stat3 and Smad1/5/8 was significantly increased (FIG. 3B). Activation of Stat3 signaling in Il-6−/− mice is probably due to LPS-induced mediators of the IL-6 family such as leukemia inhibitory factor (LIF), IL-11, or oncostatin M, already described as an hepcidin inducer (Chung et al.). Up-regulation of Inhbb mRNA together with activation of Smad1/5/8 signaling in these mice also suggests a role for activin B in signal transduction.

Activin B induces hepcidin expression and SMAD1/5/8 phosphorylation in human hepatoma-derived cells.

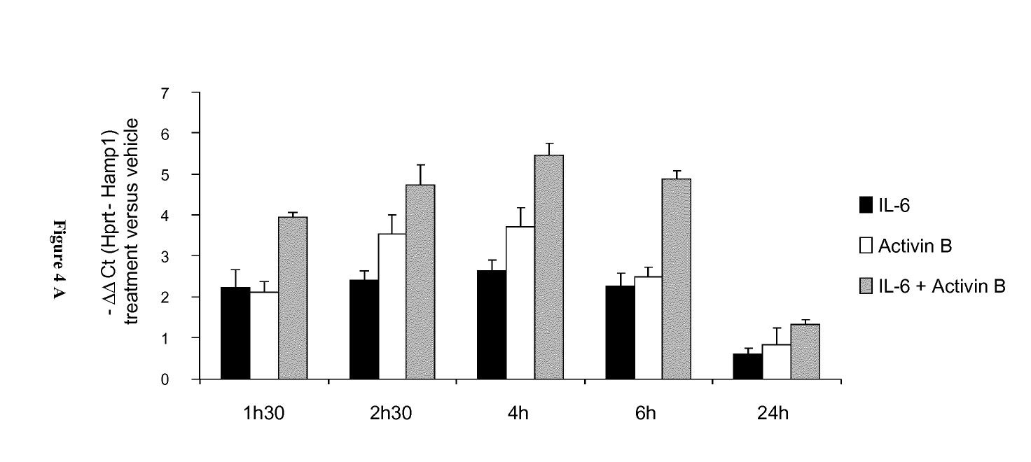

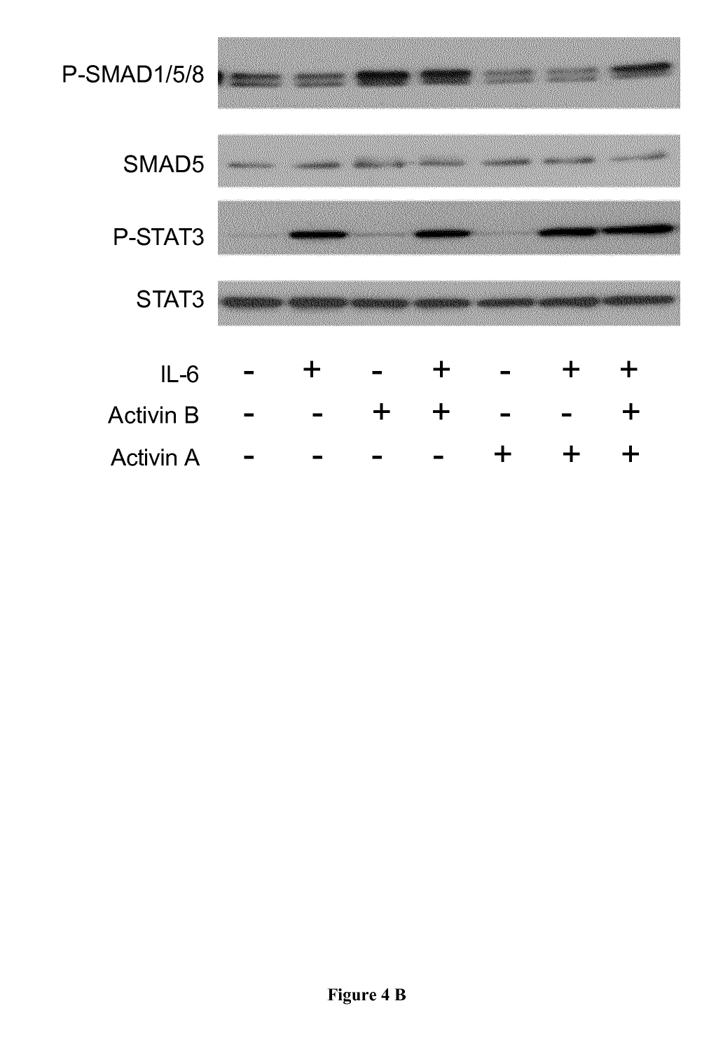

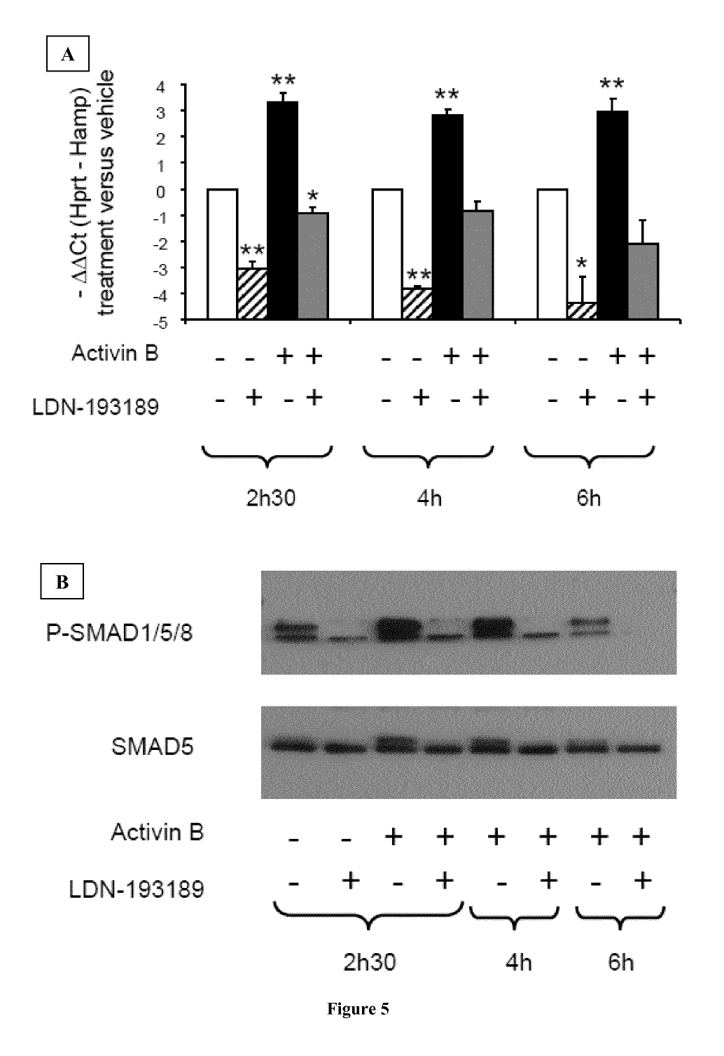

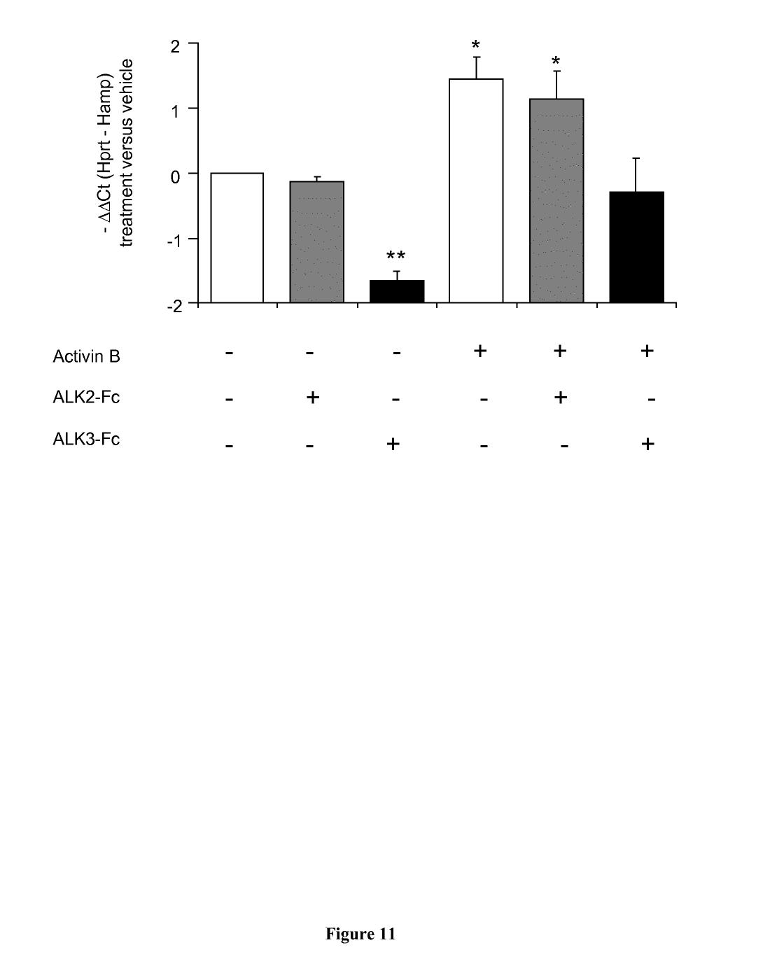

We next examined the role of activin B, alone or in combination with IL-6, on hepcidin expression in the human hepatoma-derived HepG2 cells. Cells were treated with IL-6, activin B, or both, for periods of time ranging from 1 h30 to 24 h. We found that activin B increased hepcidin mRNA expression about 13 fold (−ΔΔCt=3.71±0.46), with peak levels achieved 4 hours after treatment. Its effect on hepcidin upregulation is larger than that of IL-6 (only about 6 fold; −ΔΔCt=2.63±0.26) and, interestingly, the combination of activin B and IL-6 increased hepcidin mRNA levels much more (over 43 fold; −ΔΔCt=5.44±0.32) than the additive effects of the 2 molecules (FIG. 4A). We then evaluated the ability of activin B, alone or in combination with IL-6, to activate different signaling pathways in HepG2 cells after 2 h30, 4 h or 6 h of treatment. As expected, IL-6 induces STAT3 phosphorylation. However, more interestingly, activin B induces the phosphorylation of the SMAD effectors 1, 5 and 8 after an exposure of 2 h30 (FIG. 4B), 4, or 6 hours (data not shown). Noteworthy, the two signaling pathways are independent of each other and the capacity of activin B to induce SMAD1/5/8 phosphorylation is not shared with activin A (FIG. 4B). These data suggest that activin B could bind to BMP type I receptors rather than to the conventional activin type I receptors ALK4 and ALK7 which have so far been reported to signal only through Smad2 and Smad3. To check this possibility, we treated HepG2 cells with the BMP type I receptor inhibitor LDN-193189 (100 nM) 30 min before adding activin B. At this concentration, LDN-193189 was shown to inhibit activity of BMP type I receptors but not that of activin and TGF-β type I receptors. As shown on FIG. 5, pre-treatment with LDN-193189 completely abolished induction of hepcidin gene expression and SMAD1/5/8 phosphorylation 2 h30, 4 or 6 hours after stimulation with activin B. This demonstrates that, in human liver cells, the effect of activin B on hepcidin expression is entirely attributable to its effects on BMP signaling. We then used soluble BMP type I receptors to identify the receptor which is involved in activin B signaling. Alk1 is predominantly expressed in the endothelium and we were not able to detect Alk6 mRNA expression in the mouse liver or in HepG2 cells. We therefore focused on the two other BMP type I receptors and pretreated HepG2 cells with soluble forms of ALK2 (ALK2-Fc) or ALK3 (ALK3-Fc). As shown on FIG. 11, only ALK3-Fc prevented the induction of hepcidin gene expression by activin B, suggesting that ALK3 is the most likely type I receptor involved in activin B signaling.

Activin B also induces hepcidin expression and Smad1/5/8 phosphorylation in mouse primary hepatocytes.

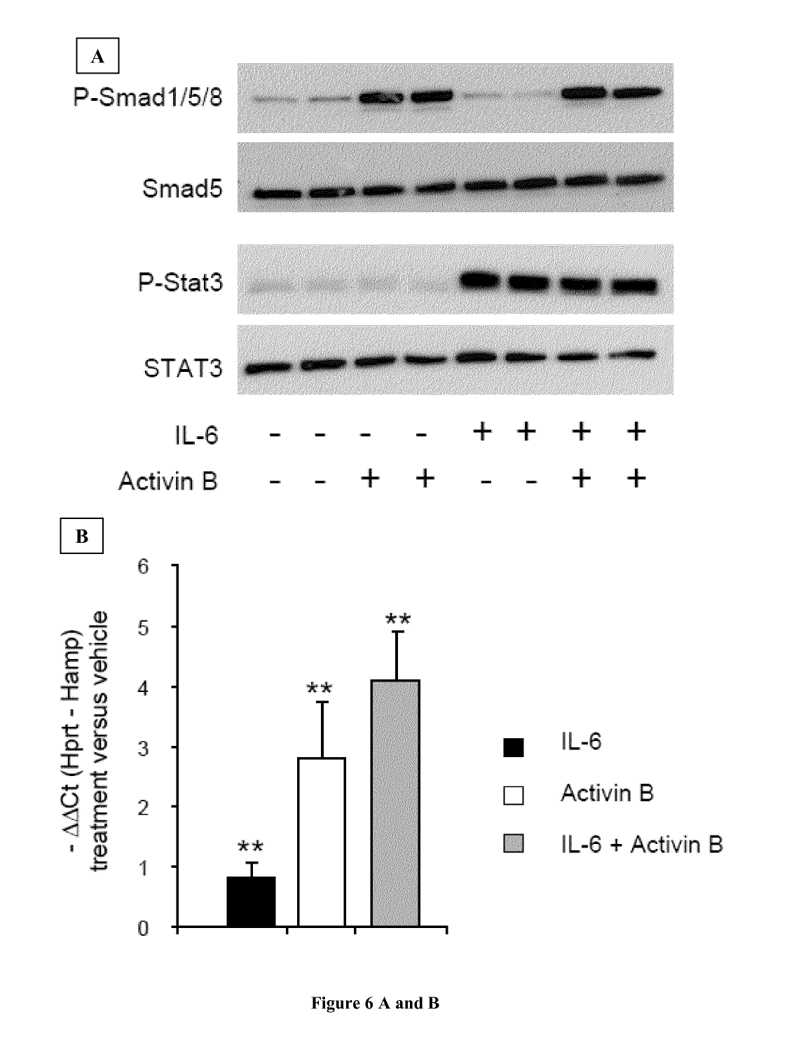

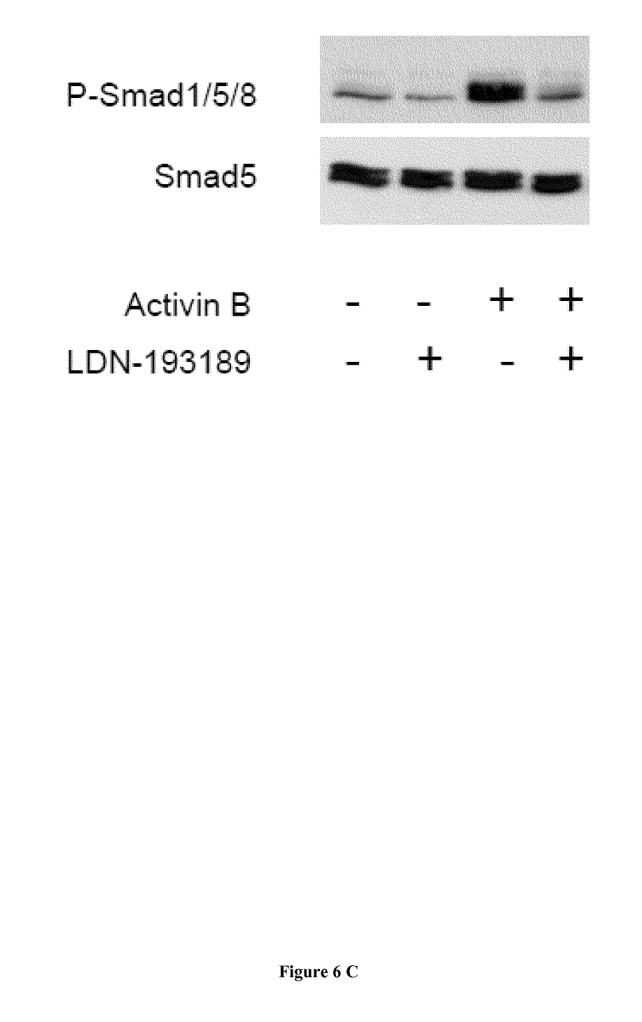

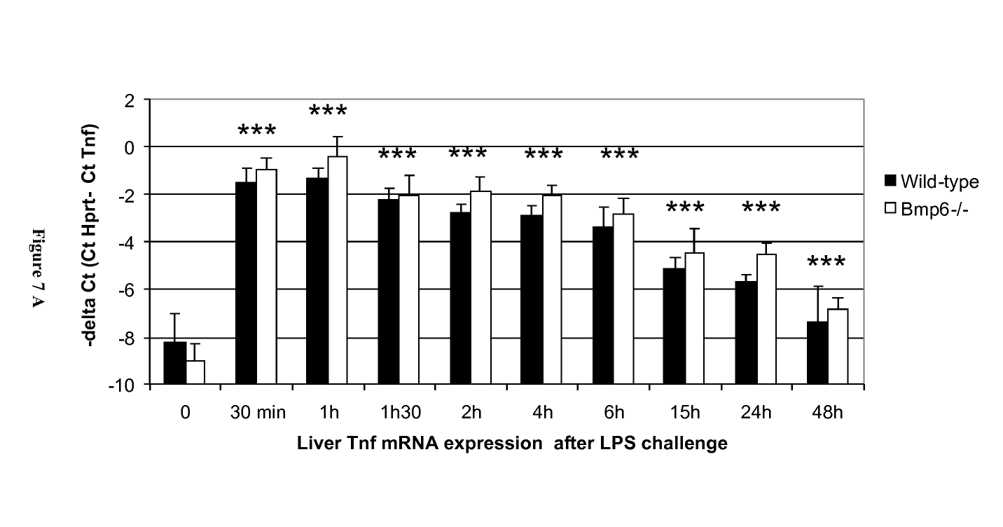

To further evaluate the impact of activin B on the regulation of hepcidin gene expression, we measured basal hepcidin expression and the ability of activin B to induce this expression in primary hepatocytes isolated from Bmp6-deficient mice. Due to the lack of functional Bmp6, these mice are insensitive to iron and are a valuable resource in identifying the molecular pathways through which hepcidin expression is upregulated specifically by inflammation. Activin B rapidly increased hepcidin mRNA expression in mouse primary hepatocytes, with peak levels achieved 2 h30 after stimulation. Hepcidin expression then remained stable up to at least 4 hours (data not shown). Interestingly, despite a strong activation of Stat3 signaling (FIG. 6A), the effect of IL-6 on hepcidin upregulation (about 1.8-fold increase; −ΔΔCt=0.82±0.24) is relatively modest (FIG. 6B), compared with that of activin B (about 7-fold increase; −ΔΔCt=2.81±0.92). There is, however, similarly to HepG2 cells, a clear synergy between Smad1/5/8 and Stat3 signaling with a 17-fold increase (−ΔΔCt=4.10±0.80) when primary hepatocytes were treated with both activin B and IL-6. Pretreatment of mouse primary hepatocytes with the BMP type I receptor inhibitor LDN-193189 prevented induction of Smad1/5/8 phosphorylation (FIG. 6C) and upregulation of Hamp gene expression by activin B, confirming that the effect of activin B on hepcidin expression is attributable to its effects on BMP signaling not only in hepatoma-derived cells but also in primary hepatocytes.

### REFERENCES

Throughout this application, various references describe the state of the art to which this invention pertains. The disclosures of these references are hereby incorporated by reference into the present disclosure.
Ganz, T. (2006). Molecular pathogenesis of anemia of chronic disease. Pediatr Blood Cancer 46, 554-557.
Ganz, T., and Nemeth, E. (2009). Iron sequestration and anemia of inflammation. Semin Hematol 46, 387-393.
Ludlow H, Muttukrishna S, Hyvönen M, Groome N P. velopment of a new antibody to the human inhibin/activin betaB subunit and its application to improved inhibin B ELISAs. J Immunol Methods. 2008 Jan. 1; 329(1-2):102-11. Epub 2007 Oct. 23.
Nemeth, E., Rivera, S., Gabayan, V., Keller, C., Taudorf, S., Pedersen, B. K., and Ganz, T. (2004a). IL-6 mediates hypoferremia of inflammation by inducing the synthesis of the iron regulatory hormone hepcidin. J Clin Invest 113, 1271-1276.
Weiss, G., and Goodnough, L. T. (2005). Anemia of chronic disease. N Engl J Med 352, 1011-1023.

1. A method of treating anemia of inflammation in a patient in need thereof, comprising the step of administering to the patient a therapeutically effective amount of a compound which inhibits the binding of activin B to ALK3 receptor or a compound which is an inhibitor of activin B expression or ALK3 receptor expression.

2. The method according to the claim 1 wherein said compound is an antibody which inhibits the binding of activin B to ALK3 receptor.

3. The method according to claim 2 wherein said antibody is an antibody against activin B.

4. The method according to claim 2 wherein said antibody is an antibody against ALK3 receptor.

5. The method according to claim 1, wherein said compound is an inhibitor of activin B expression or an inhibitor of ALK3 receptor expression.

6. The method of claim 1, wherein the anemia of inflammation is induced by an infection, a malignancy or an auto-immune disorder.

7. The method of claim 1, wherein said compound is administered as a pharmaceutical composition comprising a pharmaceutically acceptable carrier.

8. A method for diagnosis anemia of inflammation in a patient comprising:
determining the expression level of Activin B in a sample obtained from said patient; and
comparing said expression level to a threshold value.
